(19) United States
(12) Patent Application Publication
(10) Pub. No.: US 2022/0380812 A1
(43) Pub. Date: Dec. 1, 2022

(54) CRISPR/CAS9 SYSTEM AS AN AGENT FOR INHIBITION OF POLYOMA JC INFECTION

(71) Applicant: TEMPLE UNIVERSITY - OF THE COMMONWEALTH SYSTEM OF HIGHER EDUCATION, Philadelphia, PA (US)

(72) Inventors: Kamel KHALILI, Bala Cynwyd, PA (US); Hassen S. WOLLEBO, Philadelphia, PA (US); Jennifer GORDON, Philadelphia, PA (US); Ilker K. SARIYER, Broomall, PA (US)

(21) Appl. No.: 17/776,120

(22) PCT Filed: Nov. 11, 2020
(86) PCT No.: PCT/US20/59945

§ 371 Date: May 11, 2022

Related U.S. Application Data
(60) Provisional application No. 62/933,929, filed on Nov. 11, 2019.

(51) Int. Cl. C12N 15/90; C12N 9/22; C12N 15/11

(52) U.S. Cl. CPC C12N 15/907; C12N 9/22; C12N 15/11; C12N 2800/80; C12N 2310/20; A61K 38/00

(57) ABSTRACT

Provided herein are gene editing compositions and methods that effectively modulate and/or edit a JCV genome. The effective modulation and/or editing is, in an aspect, achieved by gene editing compositions targeting a NCCR region, an early coding gene, and/or a late coding gene.

### CROSS-REFERENCE

This application claims the benefit of U.S. Provisional Application No. 62/933,929, filed Nov. 11, 2019, which application is incorporated herein by reference.

### BACKGROUND

Progressive multifocal leukoencephalopathy (PML) is a demyelinating disease of the brain caused by the JC virus (JCV). Prompt intervention is important for PML because PML has a 3-month mortality rate of 20-50%. Generally, reconstitution or activation of the immune system yields the best prognosis for treating PML. However, immune system reconstitution therapy can lead to undesired effect, such as, immune reconstitution inflammatory syndrome (IRIS). Thus, an effective therapy is needed for PML.

### SUMMARY

Disclosed herein, in certain embodiments, are compositions comprising: (a) a Clustered Regularly Interspaced Short Palindromic Repeat (CRISPR)-associated endonuclease or a nucleic acid sequence encoding the CRISPR-associated endonuclease; (b) a first guide RNA (gRNA) or a nucleic acid sequence encoding the first gRNA, the first gRNA being complementary to a first target nucleic acid sequence within or near a non-coding control region (NCCR) of a John Cunningham virus (JCV) genome; and (c) a second gRNA or a nucleic acid sequence encoding the second gRNA, the second gRNA being complementary to a second target nucleic acid sequence within or near a T antigen gene of the JCV genome or a VP gene of the JCV genome. In some embodiments, the second gRNA is complementary to a second target nucleic acid sequence within or near the T antigen gene of the JCV genome. In some embodiments, the compositions further comprise a third gRNA or a nucleic acid sequence encoding the third gRNA, the third gRNA being complementary to a third target nucleic acid sequence within or near the VP gene of the JCV genome. In some embodiments, the second gRNA is complementary to a second target nucleic acid sequence within or near the VP gene of the JCV genome. In some embodiments, the CRISPR-associated endonuclease is Type I, Type II, or Type III Cas endonuclease. In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 nuclease. In some embodiments, the Cas9 nuclease is a Staphylococcus aureus Cas9 nuclease. In some embodiments, the T antigen gene is a Large T antigen gene. In some embodiments, the VP gene is a VP1 gene, VP2 gene, or a VP3 gene. In some embodiments, the VP gene is a VP1 gene. In some embodiments, the first target nucleic acid sequence comprises a sequence comprising at least about 90% sequence identity to SEQ ID NO: 2. In some embodiments, the first target nucleic acid sequence comprises a sequence according to SEQ ID NO: 2. In some embodiments, the first target nucleic acid sequence comprises a PAM sequence comprising at least about 90% sequence identity to SEQ ID NO: 3. In some embodiments, the first target nucleic acid sequence comprises a PAM sequence according to SEQ ID NO: 3. In some embodiments, the first target nucleic acid sequence comprises a sequence comprising at least about 90% sequence identity to SEQ ID NO: 4. In some embodiments, the first target nucleic acid sequence comprises a sequence according to SEQ ID NO: 4. In some embodiments, the first gRNA is encoded by a sequence comprising at least about 90% sequence identity to SEQ ID NO: 2. In some embodiments, the first gRNA is encoded by a sequence according to SEQ ID NO: 2. In some embodiments, the first gRNA comprises a RNA sequence comprising at least about 90% sequence identity to SEQ ID NO: 5. In some embodiments, the first gRNA comprises a RNA sequence a sequence according to SEQ ID NO: 5. In some embodiments, the second target nucleic acid sequence comprises a sequence comprising at least about 90% sequence identity to SEQ ID NO: 6. In some embodiments, the second target nucleic acid sequence comprises a sequence according to SEQ ID NO: 6. In some embodiments, the second target nucleic acid sequence comprises a PAM sequence comprising at least about 90% sequence identity to SEQ ID NO: 7. In some embodiments, the second target nucleic acid sequence comprises a PAM sequence according to SEQ ID NO: 7. In some embodiments, the second target nucleic acid sequence comprises a sequence comprising at least about 90% sequence identity to SEQ ID NO: 6. In some embodiments, the second target nucleic acid sequence comprises a sequence according to SEQ ID NO: 6. In some embodiments, the second gRNA is encoded by a sequence comprising at least about 90% sequence identity to SEQ ID NO: 6. In some embodiments, the second gRNA is encoded by a sequence according to SEQ ID NO: 6. In some embodiments, the second gRNA comprises a RNA sequence comprising at least about 90% sequence identity to SEQ ID NO: 9. In some embodiments, the second gRNA comprises a RNA sequence according to SEQ ID NO: 9. In some embodiments, the third target nucleic acid sequence comprises a sequence comprising at least about 90% sequence identity to SEQ ID NO: 10. In some embodiments, the third target nucleic acid sequence comprises a sequence according to SEQ ID NO: 10. In some embodiments, the third target nucleic acid sequence comprises a PAM sequence comprising at least about 90% sequence identity to SEQ ID NO: 11. In some embodiments, the third target nucleic acid sequence comprises a PAM sequence according to SEQ ID NO: 11. In some embodiments, the third target nucleic acid sequence comprises a sequence comprising at least about 90% sequence identity to SEQ ID NO: 12. In some embodiments, the third target nucleic acid sequence comprises a sequence according to SEQ ID NO: 12. In some embodiments, the third gRNA is encoded by a sequence comprising at least about 90% sequence identity to SEQ ID NO: 10. In some embodiments, the third gRNA is encoded by a sequence according to SEQ ID NO: 10. In some embodiments, the third gRNA comprises a RNA sequence comprising at least about 90% sequence identity to SEQ ID NO: 13. In some embodiments, the third gRNA comprises a RNA sequence according to SEQ ID NO: 13.

Disclosed herein, in certain embodiments, are CRISPR-Cas systems comprising: (a) a Clustered Regularly Interspaced Short Palindromic Repeat (CRISPR)-associated endonuclease; and (b) a first guide RNA (gRNA), the first gRNA being complementary to a first target nucleic acid sequence within or near a non-coding control region (NCCR) of a John Cunningham virus (JCV) genome comprising SEQ ID NO: 2 or reverse complement thereof. In some embodiments, the CRISPR-Cas system further comprises a second gRNA being complementary to a second target nucleic acid sequence within or near a T antigen gene of the JCV genome comprising SEQ ID NO: 6. In some embodiments, the CRISPR-Cas system further comprises a third gRNA being complementary to a third target nucleic acid sequence within or near a VP1 gene of the JCV genome comprising SEQ ID NO: 10. In some embodiments, the CRISPR-Cas system further comprises a second gRNA being complementary to a second target nucleic acid sequence within or near a VP1 gene of the JCV genome comprising SEQ ID NO: 10.

Disclosed herein, in certain embodiments, are CRISPR-Cas systems comprising: (a) a Clustered Regularly Interspaced Short Palindromic Repeat (CRISPR)-associated endonuclease; and (b) a guide RNA (gRNA), the gRNA being complementary to a target nucleic acid sequence within or near a VP gene of an JCV genome comprising SEQ ID NO: 10 or reverse complement thereof.

Disclosed herein, in certain embodiments, are CRISPR-Cas systems comprising: (a) a first guide RNA (gRNA) targeting a Large T antigen gene of an JCV genome comprising SEQ ID NO: 6 or a reverse complement thereof; and (b) a second gRNA targeting a VP gene of the JCV genome comprising SEQ ID NO: 10 or a reverse complement thereof.

Disclosed herein, in certain embodiments, are nucleic acids encoding the CRISPR-Cas system described herein.

Disclosed herein, in certain embodiments, are adeno-associated virus (AAV) vectors comprising a nucleic acids encoding: (a) a Clustered Regularly Interspaced Short Palindromic Repeat (CRISPR)-associated endonuclease; (b) a first guide RNA (gRNA), the first gRNA being complementary to a first target nucleic acid sequence within or near a non-coding control region (NCCR) of a John Cunningham virus (JCV) genome; and (c) a second gRNA, the second gRNA being complementary to a second target nucleic acid sequence within or near a T antigen gene of the JCV genome or a VP gene of the JCV genome. In some embodiments, the second gRNA is complementary to a second target nucleic acid sequence within or near the T antigen gene of the JCV genome. In some embodiments, the AAV vectors further comprise a third gRNA or a nucleic acid sequence encoding the third gRNA, the third gRNA being complementary to a third target nucleic acid sequence within or near the VP gene of the JCV genome. In some embodiments, the second gRNA is complementary to a second target nucleic acid sequence within or near the VP gene of the JCV genome. In some embodiments, the CRISPR-associated endonuclease is Type I, Type II, or Type III Cas endonuclease. In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 nuclease. In some embodiments, the Cas9 nuclease is a Staphylococcus aureus Cas9 nuclease. In some embodiments, the T antigen gene is a Large T antigen gene. In some embodiments, the VP gene is a VP1 gene, VP2 gene, or a VP3 gene. In some embodiments, the VP gene is a VP1 gene. In some embodiments, the first target nucleic acid sequence comprises a sequence comprising at least about 90% sequence identity to SEQ ID NO: 2. In some embodiments, the first target nucleic acid sequence comprises a sequence according to SEQ ID NO: 2. In some embodiments, the first target nucleic acid sequence comprises a PAM sequence comprising at least about 90% sequence identity to SEQ ID NO: 3. In some embodiments, the first target nucleic acid sequence comprises a PAM sequence comprising SEQ ID NO: 3. In some embodiments, the first target nucleic acid sequence comprises a sequence comprising at least about 90% sequence identity to SEQ ID NO: 4. In some embodiments, the first target nucleic acid sequence comprises a sequence according to SEQ ID NO: 4. In some embodiments, the first gRNA is encoded by a sequence comprising at least about 90% sequence identity to SEQ ID NO: 2. In some embodiments, the first gRNA is encoded by a sequence according to SEQ ID NO: 2. In some embodiments, the first gRNA comprises a RNA sequence comprising at least about 90% sequence identity to SEQ ID NO: 5. In some embodiments, the first gRNA comprises a RNA sequence comprising SEQ ID NO: 5. In some embodiments, the second target nucleic acid sequence comprises a sequence comprising at least about 90% sequence identity to SEQ ID NO: 6. In some embodiments, the second target nucleic acid sequence comprises a sequence comprising SEQ ID NO: 6. In some embodiments, the second target nucleic acid sequence comprises a PAM sequence comprising at least about 90% sequence identity to SEQ ID NO: 7. In some embodiments, the second target nucleic acid sequence comprises a PAM sequence comprising SEQ ID NO: 7. In some embodiments, the second target nucleic acid sequence comprises a sequence comprising at least about 90% sequence identity to SEQ ID NO: 6. In some embodiments, the second target nucleic acid sequence comprises a sequence comprising SEQ ID NO: 6. In some embodiments, the second gRNA is encoded by a sequence comprising at least about 90% sequence identity to SEQ ID NO: 6. In some embodiments, the second gRNA is encoded by a sequence according to SEQ ID NO: 6. In some embodiments, the second gRNA comprises a RNA sequence comprising at least about 90% sequence identity to SEQ ID NO: 9. In some embodiments, the second gRNA comprises a RNA sequence comprising SEQ ID NO: 9. In some embodiments, the third target nucleic acid sequence comprises a sequence comprising at least about 90% sequence identity to SEQ ID NO: 10. In some embodiments, the third target nucleic acid sequence comprises a sequence comprising SEQ ID NO: 10. In some embodiments, the third target nucleic acid sequence comprises a PAM sequence comprising at least about 90% sequence identity to SEQ ID NO: 11. In some embodiments, the third target nucleic acid sequence comprises a PAM sequence comprising SEQ ID NO: 11. In some embodiments, the third target nucleic acid sequence comprises a sequence comprising at least about 90% sequence identity to SEQ ID NO: 12. In some embodiments, the third target nucleic acid sequence comprises a sequence comprising SEQ ID NO: 12. In some embodiments, the third gRNA is encoded by a sequence comprising at least about 90% sequence identity to SEQ ID NO: 10. In some embodiments, the third gRNA is encoded by a sequence according to SEQ ID NO: 10. In some embodiments, the third gRNA comprises a RNA sequence comprising at least about 90% sequence identity to SEQ ID NO: 13. In some embodiments, the third gRNA comprises a RNA sequence comprising SEQ ID NO: 13. In some embodiments, the nucleic acid further comprises a promoter. In some embodiments, the promoter is a ubiquitous promoter. In some embodiments, the promoter is a tissue-specific promoter. In some embodiments, the promoter is a constitutive promoter. In some embodiments, the promoter is a human cytomegalovirus promoter. In some embodiments, the nucleic acid further comprises an enhancer element. In some embodiments, the enhancer element is a human cytomegalovirus enhancer element. In some embodiments, the nucleic acid further comprises a 5′ ITR element and 3′ ITR element. In some embodiments, the adeno-associated virus (AAV) vector is AAV2, AAVS, AAV6, AAV7, AAV8, or AAV9. In some embodiments, the nucleic acid comprises at least about 90% sequence identity to SEQ ID NO: 14. In some embodiments, the AAV vectors is an AAV6 vector or an AAV9 vector.

Disclosed herein, in certain embodiments, are methods of excising part or all of a John Cunningham virus (JCV) sequence from a cell, the method comprising providing to the cell the composition described herein, the CRISPR-Cas system described herein, or the AAV vector described herein.

Disclosed herein, in certain embodiments, are methods of inhibiting or reducing John Cunningham virus (JCV) replication in a cell, the method comprising providing to the cell the composition described herein, the CRISPR-Cas system described herein, or the AAV vector described herein. In some embodiments, the cell is in a subject. In some embodiments, the subject is a human. In some embodiments, the JCV sequence is integrated into the cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C show schematic representations of the JCV (Mad-1) genome.

FIG. 2 shows a schematic representation of AAV6/AAV9 vectors harboring a CRISPR-Cas system.

FIG. 3 shows data confirming Cas protein expression upon AAV-mediated delivery to cells.

FIGS. 4A-4D show results from CRISPR-Cas DNA excision assays.

FIGS. 5A-5B show the efficiency of AAV6GFP and AAV9 mediated transduction into cells.

### DETAILED DESCRIPTION

Provided herein are gene editing systems (e.g. CRISPR-Cas9) useful for inhibiting, reducing, or ameliorating JCV infection. For instance, provided are methods of for inhibiting, reducing, or ameliorating JCV infection by the gene editing systems that effectively edit JCV DNA. Provided herein are programmable nucleases that target JCV viral genomic DNA that is either integrated into a host cell's genome or maintained extrachromosomally (e.g. not integrated into the genome of a host cell). For example, in some instances, targeting specific genes or elements of the JCV genome leads to the depletion of infectious viral genomes and/or excision of regions within the viral genome at coding sequences or regulatory regions to inactivate viral replication. In certain instances, there is additional benefit characterized in that the gene editing systems effectively excise integrated viral and/or pro-viral genomes. As viral sequences generally bear little sequence similarity to a host genome, the described compositions and methods of treatment can also produce fewer off-target effects than editing therapies targeting a loci endogenous to a cell.

JCV is a nonenveloped, T=7 icosahedral polyomavirus with a closed circular, supercoiled, double-stranded DNA genome. The capsid is composed of three viral structural proteins, VP1, VP2, and VP3, with VP1 being the major constituent. There are 72 pentamers, each composed of five VP1 molecules and one molecule of either VP2 or VP3. Only VP1 is exposed on the surface of the capsid, and it thus determines receptor specificity. Polyomavirus DNA is nucleosomal in structure, generally having approximately 25 nucleosomes composed of viral DNA and host cell histones contained in each mini-chromosome.

The prototype JCV genome generally consists of 5,130 bp, although individual variants differ in length, due to alterations in their noncoding regions . The genome encodes 6 major viral proteins (large T and small t antigens, VP1, VP2, VP3, and agnoprotein) as well as several splice variants of T antigen. Early- and late-transcribing sides of the genome are physically separated by a noncoding control region (NCCR), also termed the hypervariable regulatory region (HVRR) or regulatory region (RR) and are transcribed in opposite directions from opposite strands of DNA. The early side of the genome, which is transcribed before viral DNA replication begins, is composed of large T antigen and small t antigen genes, as well as certain splice variants. The late side of the genome is transcribed concomitant with DNA replication and encodes the three viral structural proteins, VP1, VP2, and VP3, as well as the accessory agnoprotein.

Polyomaviruses, including JCV, display restricted cell type specificities for lytic infection. Furthermore, JCV can establish low-level persistent or latent infections in certain cell types. Once present in the central nervous system (CNS), the virus replicates vigorously in oligodendrocytes, leading to the demyelinating disease PML. The cell type-specific tropism of JCV observed in vivo mirrors in vitro tropisms, with virus productively infecting bone marrow-derived cells, tonsillar stromal cells, and macroglia. In vivo, JCV infection is generally restricted to kidney epithelial cells, tonsillar stromal cells, bone marrow-derived cell lineages, oligodendrocytes, and astrocytes. Efficient JCV replication is maximal in primary glial cell cultures and certain human glial cell lines. While the major tropism of JCV for human glial cells is not fully understood, but multiple factors are likely responsible for contributing to robust viral replication in this cell type. For example, a diversity of host cell-specific transcription and replication factors differentially influence the restricted specificity displayed by JCV.

The gene editing systems (e.g. CRISPR-Cas9) provided herein utilize novel targets and/or combinations of targets within the JCV genome to efficiently and effectively edit the JCV DNA, thereby inhibiting, reducing, or preventing JCV infection. For example, combinations of targets including the NCCR and additional late (e.g. a VP genes) and/or early genes (e.g. a T antigen gene) yield effective editing and/or excision of the JCV genome or regions thereof, thereby yielding a replication incompetent JCV genome. Accordingly, the design and synthesis of the disclosed vectors encoding the gene editing machinery (e.g. a Cas9 endonuclease) and targeting nucleic molecules (e.g. guide RNAs corresponding to a target sequence) enable the efficient editing of JCV in cells. Furthermore, the gene editing systems described herein provide a solution for the effective and/or efficient editing of a JCV genome, integrated or non-integrated, thereby inhibiting, reducing, or ameliorating both primary and latent JCV infection in cells.

Programmable nucleases enable precise genome editing by, in some instances, introducing DNA double-strand breaks (DSBs) at specific genomic loci, thereby initiating gene editing. Generally, as embodied herein, a variety of gene editing systems can be employed to target the JCV genes, elements, or regions described herein (e.g. targeting a NCCR region and one or both of a T antigen gene and a VP gene). In some embodiments, the gene editing system comprises a CRISPR-Cas system. In some embodiments, the gene editing system comprises meganucleases. In some embodiments, the gene editing system comprises zinc finger nucleases (ZFNs). In some embodiments, the gene editing system comprises transcription activator—like effector nucleases (TALENs). These gene editing systems can be broadly classified into two categories based on their mode of DNA recognition: ZFNs, TALENs and meganucleases achieve specific DNA binding via protein-DNA interactions, whereas CRISPR-Cas systems are targeted to specific DNA sequences by a short RNA guide molecule that base-pairs directly with the target DNA and by protein-DNA interactions. Accordingly, protein targeting or nucleic acid targeting can be employed to target the JCV DNA loci described herein.

For example, described and provided herein are CRISPR-Cas compositions and methods useful for reducing or inhibiting JCV replication within a cell. By way of further example, the described and provided CRISPR-Cas compositions are useful for modulating (e.g. altering, removing, etc.) and/or editing (e.g. excising) genomic JCV DNA in a cell, thereby inhibiting JCV replication. Inhibiting JCV by modulating and/or editing JCV DNA in a cell yields an incompetent and/or deficient JCV genome structure that does not allow for replication of the JCV virus. As such, the CRISPR-Cas compositions and methods described herein can be used to effectively modulate and/or edit a JCV genome in a cell. In some embodiments, the cells already comprise a JCV infection. In some embodiments, the CRISPR systems provided herein eliminate or reduce a latently infected cell. In some embodiments, the CRISPR systems provided herein modulate and/or edit JCV DNA in a latently infected cell. In some embodiments, the CRISPR systems provided herein prevent JCV infection. Cells of interests are cells susceptible to JCV infection (see, for example, Molecular Biology, Epidemiology, and Pathogenesis of Progressive Multifocal Leukoencephalopathy, the JC Virus-Induced Demyelinating Disease of the Human Brain. Michael W. Ferenczy et. al, Clinical Microbiology Reviews Jul 2012, 25 (3) 471-506). Such cells include, but are not limited to, cells within the nervous system, such as glial cells (e.g. oligodendrocytes, astrocytes, etc.).

### Cas Nucleases

Engineered CRISPR systems generally contain two components: a guide RNA (gRNA or sgRNA) and a CRISPR-associated endonuclease (Cas protein). In nature, CRISPR/CRISPR-associated (Cas) systems provide bacteria and archaea with adaptive immunity against viruses and plasmids by using CRISPR RNAs (crRNAs) to guide the silencing of invading nucleic acids. The CRISPR-Cas is a RNA-mediated adaptive defense system that relies on small RNA molecules for sequence-specific detection and silencing of foreign nucleic acids. CRISPR/Cas systems are composed of cas genes organized in operon(s) and CRISPR array(s) consisting of genome-targeting sequences (called spacers). Provided herein are engineered CRISPR systems that detect and silence JCV DNA in a cell.

As described herein, CRISPR-Cas systems generally refer to an enzyme system that includes a guide RNA sequence that contains a nucleotide sequence complementary or substantially complementary to a region of a target polynucleotide (e.g. JCV genomic DNA), and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, CRISPR-Cas systems further comprise transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. In certain embodiments, a target sequence comprises any polynucleotide, such as DNA or RNA polynucleotides. In some embodiments, a target sequence is located in the nucleus or cytoplasm of a cell.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ.

In some embodiments, the Cas9 protein can be from or derived from: Staphylococcus aureus, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the composition comprises a CRISPR-associated (Cas) protein, or functional fragment or derivative thereof. In some embodiments, the Cas protein is an endonuclease, including but not limited to the Cas9 nuclease. In some embodiments, the Cas9 protein comprises an amino acid sequence identical to the wild type Streptococcus pyogenes or Staphylococcus aureus Cas9 amino acid sequence. In some embodiments, the Cas protein comprises the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas proteins, useful for the present disclosure, known or can be identified, using methods known in the art (see e.g., Esvelt et al., 2013, Nature Methods, 10: 1116-1121). In some embodiments, the Cas protein comprises a modified amino acid sequence, as compared to its natural source.

CRISPR/Cas proteins comprise at least one RNA recognition and/or RNA binding domain. RNA recognition and/or RNA binding domains interact with guide RNAs (gRNAs). CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

In some embodiments, the CRISPR/Cas-like protein can be derived from a wild type Cas protein or fragment thereof. In some embodiments, the CRISPR/Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, domains of the Cas9 protein not involved in RNA-guided cleavage can be eliminated from the protein such that the modified Cas9 protein is smaller than the wild-type Cas9 protein.

The disclosed CRISPR-Cas compositions should also be construed to include any form of a protein having substantial homology to a Cas protein (e.g., Cas9, saCas9, Cas9 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein.

### JCV Targeting

The CRISPR-Cas systems described herein achieve, in some embodiments, the effective modulation and/or editing of JCV DNA (e.g. a JCV genome) through use of gRNA targets and combinations of gRNA targets. The gRNA targets described target novel sequences and/or regions of the JCV genome wherein, in some embodiments, the target sequences and/or regions confer a benefit by enabling effective editing of a JCV DNA molecule and/or the JCV genome in a cell. Additionally, in another aspect, further advantage is provided by use of the novel target sequences in combination. For example, gRNAs described herein target the non-coding control region (NCCR) of the JCV genome, a late coding gene, and/or an early coding gene to modulate and/or edit JCV DNA and prevent/inhibit JCV replication. In certain embodiments, targeting includes promoting or inducing any editing (e.g. excising, deleting, etc.) of the NCCR region, large T antigen gene, and/or VP1 gene. In certain embodiments,

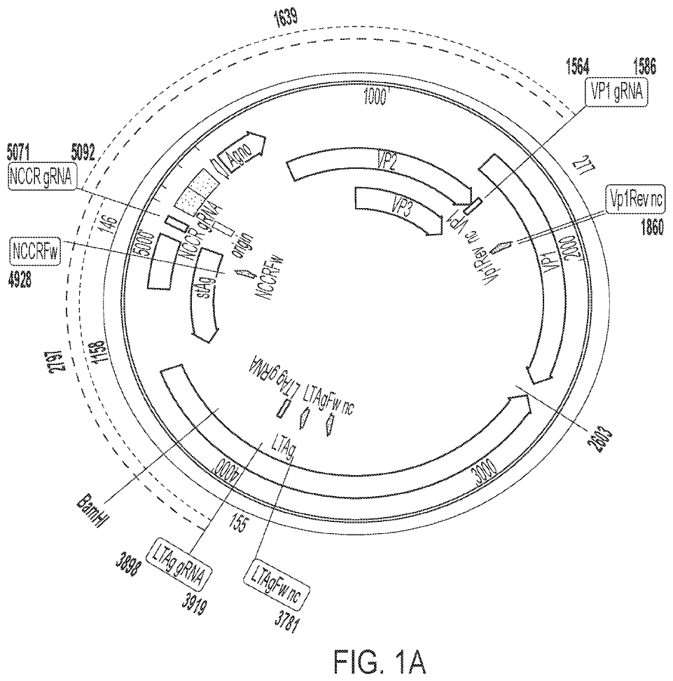

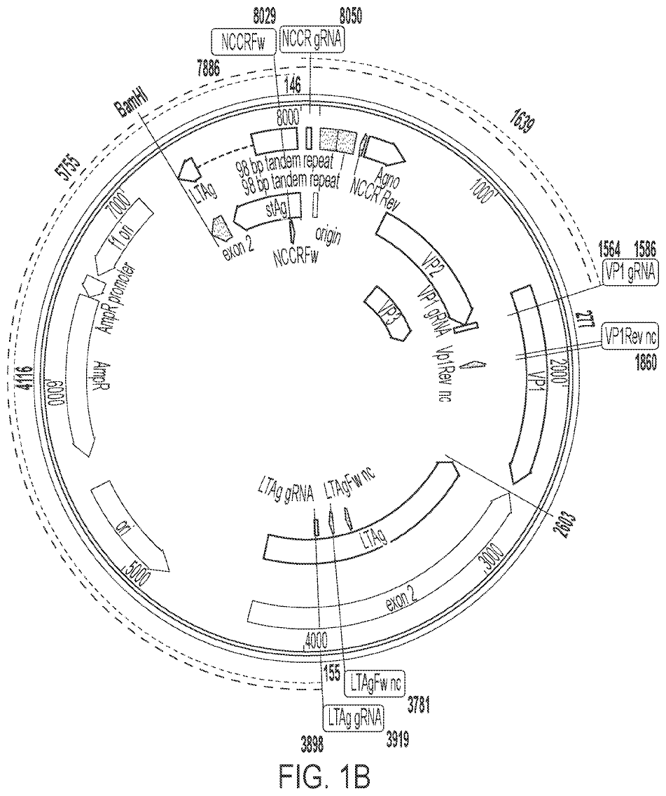

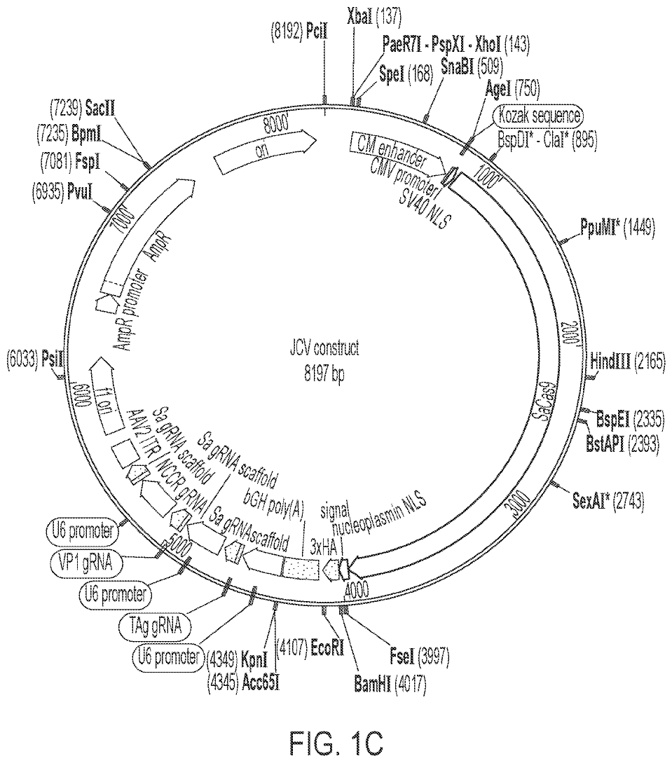

The JCV genome is a closed circular supercoiled chromosome that is composed of “early” and “late” genes that are separated by the non-coding control region (NCCR), which contains the origin of replication (ORI), promoter, and enhancer elements. The early region is expressed de novo after infection and before DNA replication and is on the ORI-proximal side of the NCCR. Late genes are optimally expressed concurrently with or after DNA replication and are found on the ORI-distal side of the NCCR. Polyomavirus NCCRs are the most variable portions of the viral genome within a single virus as well as across genera of viruses. Notably, the gRNAs disclosed herein target a region proximally 5′ (or distally 3′ considering that that the JCV genome is circular) (e.g. within positions about 5000 to about 5130 of the Mad-1 genome) relative to the position the sequence block elements and tandem repeat elements of the JCV genome. FIG. 1 shows a diagram of the JCV (Mad-1) genome, denoting the location of the NCCR target. In some embodiments, an advantage is provided by targeting a region proximally 5′ (or distally 3′ considering that that the JCV genome is circular) (e.g. within positions about 5000 to about 5130 of the Mad-1 genome) relative to the position the sequence block elements and tandem repeat elements of the JCV genome. In some embodiments, a gRNA targets the CRISPR-Cas system to a NCCR region of the JCV genome. In some embodiments, a gRNA targets the CRISPR-Cas system to a sequence of the JCV genome comprising SEQ ID NO: 2. In some embodiments, a first gRNA targeting the NCCR is used combination with gRNA targeting one or more coding regions (e.g. Large T and small t antigens, VP1, VP2, VP3, and the agnoprotein).

The JCV genome encodes 6 major viral proteins (large T and small t antigens, VP1, VP2, VP3, and agnoprotein) as well as several splice variants of T antigen. Early- and late-transcribing sides of the genome are physically separated by the noncoding control region (NCCR), often called the hypervariable regulatory region (HVRR) or regulatory region (RR) and are transcribed in opposite directions from opposite strands of DNA. The early coding genes of the genome, which is transcribed before DNA replication begins, is composed of large T antigen and small t antigen genes, as well as the T antigen splice variants. The late coding genes of the genome is transcribed concomitant with DNA replication and encodes the three viral structural proteins, VP1, VP2, and VP3, as well as the accessory agnoprotein. In some embodiments, a gRNA targets the CRISPR-Cas system to an early coding gene. In some certain embodiments, the early coding gene is a T antigen gene. In some certain embodiments, the T antigen is a Large T antigen gene. In some embodiments, the gRNA targets the CRISPR-Cas system to a sequence of the JCV genome comprising SEQ ID NO: 6. In some embodiments, a gRNA targets the CRISPR-Cas system to a late coding gene. In some certain embodiments, the early coding gene is a VP gene. In some certain embodiments, the VP gene is a VP1 gene. In some embodiments, the gRNA targets the CRISPR-Cas system to a sequence of the JCV genome comprising SEQ ID NO: 10.

Generally, a target sequence comprises a protospacer adjacent motif (PAM). In some instances, a PAM refers to a DNA sequence required for a Cas/sgRNA to form an R-loop to interrogate a specific DNA sequence through Watson-Crick pairing of its guide RNA with the genome. In certain instances, the PAM specificity is a function of the DNA-binding specificity of the Cas protein (e.g., a PAM recognition domain of a Cas), wherein, a protospacer adjacent motif recognition domain, in some instances, refers to a Cas amino acid sequence that comprises a binding site to a DNA target PAM sequence. In the CRISPR-Cas system derived from S. pyogenes (spCas9), the target DNA typically immediately precedes a 5′-NGG or NAG proto-spacer adjacent motif (PAM). Other Cas9 orthologs can have different PAM specificities. For example, Cas9 from S. thermophilus (stCas9) requires 5′-NNAGAA for CRISPR 1 and 5′-NGGNG for CRISPR3 and Neiseria menigiditis (nmCas9) requires 5′-NNNNGATT. Cas9 from Staphylococcus aureus subsp. aureus (saCas9) requires 5′-NNGRRT (R=A or G). The JCV targets provided herein can further be identified in the context of the gRNAs.

The gRNA is a short RNA nucleotide spacer that defines the genomic target to be modified. As used herein, the term guide RNA (gRNA or sgRNA) refers to a RNA containing a sequence that corresponds and/or hybridizes to a target JCV sequence. For example, SEQ ID NO: 2 comprises a JCV target sequence wherein the gRNA comprises SEQ ID NO: 5, the RNA equivalent of target sequence. The guide RNA sequence can be a sense or anti-sense sequence. A guide RNA can, in some embodiments, include nucleotide sequences other than the region complementary or substantially complementary to a region of a target DNA sequence. For example, in some instances, a guide RNA is part or considered part of a crRNA or an included in a crRNA, e.g., a crRNA:tracrRNA chimera. As used herein, a term target nucleic acid is intended to mean a nucleic acid that is the object of an action (e.g. editing or modulation).

In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalently couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments all of the inter-nucleoside linkers of the gRNA, or contiguous nucleotide sequence thereof, are modified. For example, in some embodiments the inter-nucleoside linkage comprises Sulphur (S), such as a phosphorothioate inter-nucleoside linkage.

Modifications to the ribose sugar or nucleobase can also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region (e.g. the NCCR of the JCV genome) and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence.

gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the crRNA or tracrRNA comprises a modified sequence. In certain embodiments, the crRNA or tracrRNA comprises at least 1, 2, 3, 4, 5, 10, or 15 modified bases (e.g. a modified native base sequence).

Complementary, as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double-stranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80%, 85%, 90%, or 95% sequence identity. In certain embodiments, sequence identity includes in addition to non-identical nucleobases, sequences comprising insertions and/or deletions.

In some embodiments, the composition comprises multiple different gRNA molecules, each targeted to a different target sequence. In some embodiments, this multiplexed strategy provides for increased efficacy. These multiplex gRNAs or combination of gRNAs can be expressed separately in different vectors or expressed in one single vector.

In some embodiments, the gRNA comprises a CRISPR RNA (crRNA):trans activating cRNA (tracrRNA) duplex. In some embodiments, the gRNA comprises a stem-loop that mimics the natural duplex between the crRNA and tracrRNA. In some embodiments, the stem-loop comprises a nucleotide sequence comprising AGAAAU. For example, in some embodiments, the composition comprises a synthetic or chimeric guide RNA comprising a crRNA, stem, and tracrRNA. In some embodiments, the composition comprises an isolated crRNA and/or an isolated tracrRNA which hybridize to form a natural duplex. For example, in some embodiments, the gRNA comprises a crRNA or crRNA precursor (pre-crRNA) comprising a targeting sequence.

Described herein are gRNAs targeting (e.g. hybridizing or annealing to) the NCCR, VP1 gene, Large T antigen gene, or combinations thereof. In some embodiments, the gRNAs target the NCCR, VP1 gene, and Large T antigen gene. In certain embodiments, the gRNAs targeting the NCCR, VP1 gene, and/or Large T antigen gene hybridize to a region within or near the NCCR, VP1 gene, and/or Large T antigen gene. In certain embodiments, a region within the NCCR, VP1 gene, and/or Large T antigen gene includes at least one nucleotide within the NCCR, VP1 gene, and/or Large T antigen gene. In certain embodiments, a region near the NCCR, VP1 gene, and/or Large T antigen gene includes 5, 10, 15, 20, 25, 30, or 35 base positions surrounding the NCCR, VP1 gene, and/or Large T antigen gene.

In some embodiments, the gRNA targets the NCCR of the JCV genome. In some embodiments, the gRNA targets a NCCR sequence corresponding to SEQ ID NO: 2 or a sequence having at least 90%, 95%, 97%, 98%, or 99% sequence identity to SEQ ID NO: 2. In some embodiments, the target nucleic acid sequence within or near the NCCR comprises a sequence according to SEQ ID NO: 2 or a sequence having at least 90%, 95%, 97%, 98%, or 99% sequence identity to SEQ ID NO: 2. In some embodiments, the gRNA comprises a RNA sequence of SEQ ID NO: 5 or a sequence having at least 90%, 95%, 97%, 98%, or 99% sequence identity to SEQ ID NO: 5. In some embodiments, the gRNA targets a NCCR sequence complementary to SEQ ID NO: 2 or a sequence having at least 90%, 95%, 97%, 98%, or 99% sequence identity to the NCCR sequence complementary to SEQ ID NO: 2. In some embodiments, the NCCR sequence of the NCCR targeted by the gRNA comprises a PAM sequence according to SEQ ID NO: 3 or a sequence having at least 70%, 80%, or 90% sequence identity to SEQ ID NO: 3. In some embodiments, a NCCR sequence of the NCCR targeted by the gRNA comprises SEQ ID NO: 4 or a sequence having at least 90%, 95%, 97%, 98%, or 99% sequence identity to SEQ ID NO: 4.

In some embodiments, the gRNA targets the T antigen gene of the JCV genome. In some embodiments, the gRNA targets a T antigen sequence corresponding to SEQ ID NO: 6 or a sequence having at least 90%, 95%, 97%, 98%, or 99% sequence identity to SEQ ID NO: 6. . In some embodiments, the target nucleic acid sequence within or near the T antigen gene comprises a sequence according to SEQ ID NO: 6 or a sequence having at least 90%, 95%, 97%, 98%, or 99% sequence identity to SEQ ID NO: 6. In some embodiments, the gRNA comprises a RNA sequence of SEQ ID NO: 9 or a sequence having at least 90%, 95%, 97%, 98%, or 99% sequence identity to SEQ ID NO: 9. In some embodiments, the gRNA targets a T antigen sequence complementary to SEQ ID NO: 6 or a sequence having at least 90%, 95%, 97%, 98%, or 99% sequence identity to the T antigen sequence complementary to SEQ ID NO: 6. In some embodiments, a T antigen sequence of the T antigen gene targeted by the gRNA comprises a PAM sequence comprising SEQ ID NO: 7 or a sequence having at least 70%, 80%, or 90% sequence identity to SEQ ID NO: 7. In some embodiments, the T antigen sequence of the T antigen gene targeted by the gRNA comprises SEQ ID NO: 6 or a sequence having at least 90%, 95%, 97%, 98%, or 99% sequence identity to SEQ ID NO: 6.

In some embodiments, the gRNA hybridizes to the NCCR of the JCV genome. In some embodiments, the gRNA hybridizes to a NCCR sequence corresponding to SEQ ID NO: 2 or a sequence having at least 90%, 95%, 97%, 98%, or 99% sequence identity to SEQ ID NO: 2. In some embodiments, the target nucleic acid sequence within or near the NCCR comprises a sequence according to SEQ ID NO: 2 or a sequence having at least 90%, 95%, 97%, 98%, or 99% sequence identity to SEQ ID NO: 2. In some embodiments, the gRNA comprises a RNA sequence of SEQ ID NO: 5 or a sequence having at least 90%, 95%, 97%, 98%, or 99% sequence identity to SEQ ID NO: 5. In some embodiments, the gRNA hybridizes to a NCCR sequence complementary to SEQ ID NO: 2 or a sequence having at least 90%, 95%, 97%, 98%, or 99% sequence identity to the NCCR sequence complementary to SEQ ID NO: 2. In some embodiments, the NCCR sequence of the NCCR targeted by the gRNA comprises a PAM sequence according to SEQ ID NO: 3 or a sequence having at least 70%, 80%, or 90% sequence identity to SEQ ID NO: 3. In some embodiments, a NCCR sequence of the NCCR targeted by the gRNA comprises SEQ ID NO: 4 or a sequence having at least 90%, 95%, 97%, 98%, or 99% sequence identity to SEQ ID NO: 4.

In some embodiments, the gRNA hybridizes to the T antigen gene of the JCV genome. In some embodiments, the gRNA hybridizes to a T antigen sequence corresponding to SEQ ID NO: 6 or a sequence having at least 90%, 95%, 97%, 98%, or 99% sequence identity to SEQ ID NO: 6. . In some embodiments, the target nucleic acid sequence within or near the T antigen gene comprises a sequence according to SEQ ID NO: 6 or a sequence having at least 90%, 95%, 97%, 98%, or 99% sequence identity to SEQ ID NO: 6. In some embodiments, the gRNA comprises a RNA sequence of SEQ ID NO: 9 or a sequence having at least 90%, 95%, 97%, 98%, or 99% sequence identity to SEQ ID NO: 9. In some embodiments, the gRNA hybridizes to a T antigen sequence complementary to SEQ ID NO: 6 or a sequence having at least 90%, 95%, 97%, 98%, or 99% sequence identity to the T antigen sequence complementary to SEQ ID NO: 6. In some embodiments, a T antigen sequence of the T antigen gene targeted by the gRNA comprises a PAM sequence comprising SEQ ID NO: 7 or a sequence having at least 70%, 80%, or 90% sequence identity to SEQ ID NO: 7. In some embodiments, the T antigen sequence of the T antigen gene targeted by the gRNA comprises SEQ ID NO: 6 or a sequence having at least 90%, 95%, 97%, 98%, or 99% sequence identity to SEQ ID NO: 6.

In some embodiments, the gRNA hybridizes to a VP gene of the JCV genome. In some embodiments, the gRNA hybridizes to a VP sequence corresponding to SEQ ID NO: 10 or a sequence having at least 90%, 95%, 97%, 98%, or 99% sequence identity to SEQ ID NO: 10. In some embodiments, the target nucleic acid sequence within or near the VP gene comprises a sequence according to SEQ ID NO: 10 or a sequence having at least 90%, 95%, 97%, 98%, or 99% sequence identity to SEQ ID NO: 10. In some embodiments, the gRNA comprises a RNA sequence of SEQ ID NO: 9 or a sequence having at least 90%, 95%, 97%, 98%, or 99% sequence identity to SEQ ID NO: 9. In some embodiments, the gRNA hybridizes to a VP sequence complementary to SEQ ID NO: 10 or a sequence having at least 90%, 95%, 97%, 98%, or 99% sequence identity to the VP sequence complementary to SEQ ID NO: 10. In some embodiments, a VP sequence of the VP gene targeted by the gRNA comprises a PAM sequence comprising SEQ ID NO: 10 or a sequence having at least 70%, 80%, or 90% sequence identity to SEQ ID NO: 10. In some embodiments, a VP sequence of the VP gene targeted by the gRNA comprises SEQ ID NO: 10 or a sequence having at least 90%, 95%, 97%, 98%, or 99% sequence identity to SEQ ID NO: 10.

In some embodiments, the gRNA targets a VP gene of the JCV genome. In some embodiments, the gRNA targets a VP sequence corresponding to SEQ ID NO: 10 or a sequence having at least 90%, 95%, 97%, 98%, or 99% sequence identity to SEQ ID NO: 10. In some embodiments, the target nucleic acid sequence within or near the VP gene comprises a sequence according to SEQ ID NO: 10 or a sequence having at least 90%, 95%, 97%, 98%, or 99% sequence identity to SEQ ID NO: 10. In some embodiments, the gRNA comprises a RNA sequence of SEQ ID NO: 9 or a sequence having at least 90%, 95%, 97%, 98%, or 99% sequence identity to SEQ ID NO: 9. In some embodiments, the gRNA targets a VP sequence complementary to SEQ ID NO: 10 or a sequence having at least 90%, 95%, 97%, 98%, or 99% sequence identity to the VP sequence complementary to SEQ ID NO: 10. In some embodiments, a VP sequence of the VP gene targeted by the gRNA comprises a PAM sequence comprising SEQ ID NO: 10 or a sequence having at least 70%, 80%, or 90% sequence identity to SEQ ID NO: 10. In some embodiments, a VP sequence of the VP gene targeted by the gRNA comprises SEQ ID NO: 10 or a sequence having at least 90%, 95%, 97%, 98%, or 99% sequence identity to SEQ ID NO: 10.

In some embodiments, the composition comprises a nucleic acid encoding the CRISPR-associated endonuclease, said gRNA, and one or both of the gRNA and gRNA. In some embodiments, the gRNA is encoded by SEQ ID NO: 2 or a sequence having at least 90%, 95%, 97%, 98%, or 99% sequence identity to SEQ ID NO: 2. In some embodiments, the gRNA is encoded by SEQ ID NO: 6 or a sequence having at least 90%, 95%, 97%, 98%, or 99% sequence identity to SEQ ID NO: 6. In some embodiments, the gRNA is encoded by SEQ ID NO: 10 or a sequence having at least 90%, 95%, 97%, 98%, or 99% sequence identity to SEQ ID NO: 10. In some embodiments, the nucleic acid encodes both the gRNA and the gRNA.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity can be determined by procedures which are well-known in the art, for example, a BLAST program (Basic Local Alignment Search Tool at the National Center for Biological Information).

In some embodiments, the compositions described herein utilize about 1 gRNA to about 6 gRNAs. In some embodiments, the compositions described herein utilize at least about 1 gRNA. In some embodiments, the compositions described herein utilize at most about 6 gRNAs. In some embodiments, the compositions described herein utilize about 1 gRNA to about 2 gRNAs, about 1 gRNA to about 3 gRNAs, about 1 gRNA to about 4 gRNAs, about 1 gRNA to about 5 gRNAs, about 1 gRNA to about 6 gRNAs, about 2 gRNAs to about 3 gRNAs, about 2 gRNAs to about 4 gRNAs, about 2 gRNAs to about 5 gRNAs, about 2 gRNAs to about 6 gRNAs, about 3 gRNAs to about 4 gRNAs, about 3 gRNAs to about 5 gRNAs, about 3 gRNAs to about 6 gRNAs, about 4 gRNAs to about 5 gRNAs, about 4 gRNAs to about 6 gRNAs, or about 5 gRNAs to about 6 gRNAs. In some embodiments, the compositions described herein utilize about 1 gRNA, about 2 gRNAs, about 3 gRNAs, about 4 gRNAs, about 5 gRNAs, or about 6 gRNAs.

In some embodiments, the gRNA comprises about 15 nucleotides to about 28 nucleotides. In some embodiments, the gRNA comprises at least about 15 nucleotides. In some embodiments, the gRNA comprises at most about 28 nucleotides. In some embodiments, the gRNA comprises about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 15 nucleotides to about 21 nucleotides, about 15 nucleotides to about 22 nucleotides, about 15 nucleotides to about 23 nucleotides, about 15 nucleotides to about 24 nucleotides, about 15 nucleotides to about 25 nucleotides, about 15 nucleotides to about 28 nucleotides, about 16 nucleotides to about 17 nucleotides, about 16 nucleotides to about 18 nucleotides, about 16 nucleotides to about 19 nucleotides, about 16 nucleotides to about 20 nucleotides, about 16 nucleotides to about 21 nucleotides, about 16 nucleotides to about 22 nucleotides, about 16 nucleotides to about 23 nucleotides, about 16 nucleotides to about 24 nucleotides, about 16 nucleotides to about 25 nucleotides, about 16 nucleotides to about 28 nucleotides, about 17 nucleotides to about 18 nucleotides, about 17 nucleotides to about 19 nucleotides, about 17 nucleotides to about 20 nucleotides, about 17 nucleotides to about 21 nucleotides, about 17 nucleotides to about 22 nucleotides, about 17 nucleotides to about 23 nucleotides, about 17 nucleotides to about 24 nucleotides, about 17 nucleotides to about 25 nucleotides, about 17 nucleotides to about 28 nucleotides, about 18 nucleotides to about 19 nucleotides, about 18 nucleotides to about 20 nucleotides, about 18 nucleotides to about 21 nucleotides, about 18 nucleotides to about 22 nucleotides, about 18 nucleotides to about 23 nucleotides, about 18 nucleotides to about 24 nucleotides, about 18 nucleotides to about 25 nucleotides, about 18 nucleotides to about 28 nucleotides, about 19 nucleotides to about 20 nucleotides, about 19 nucleotides to about 21 nucleotides, about 19 nucleotides to about 22 nucleotides, about 19 nucleotides to about 23 nucleotides, about 19 nucleotides to about 24 nucleotides, about 19 nucleotides to about 25 nucleotides, about 19 nucleotides to about 28 nucleotides, about 20 nucleotides to about 21 nucleotides, about 20 nucleotides to about 22 nucleotides, about 20 nucleotides to about 23 nucleotides, about 20 nucleotides to about 24 nucleotides, about 20 nucleotides to about 25 nucleotides, about 20 nucleotides to about 28 nucleotides, about 21 nucleotides to about 22 nucleotides, about 21 nucleotides to about 23 nucleotides, about 21 nucleotides to about 24 nucleotides, about 21 nucleotides to about 25 nucleotides, about 21 nucleotides to about 28 nucleotides, about 22 nucleotides to about 23 nucleotides, about 22 nucleotides to about 24 nucleotides, about 22 nucleotides to about 25 nucleotides, about 22 nucleotides to about 28 nucleotides, about 23 nucleotides to about 24 nucleotides, about 23 nucleotides to about 25 nucleotides, about 23 nucleotides to about 28 nucleotides, about 24 nucleotides to about 25 nucleotides, about 24 nucleotides to about 28 nucleotides, or about 25 nucleotides to about 28 nucleotides. In some embodiments, the gRNA comprises about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, or about 28 nucleotides.

Further provided herein are gene editing compositions targeting a NCCR region, an early coding gene, and/or a late coding gene. In some embodiments, targeting the disclosed regions alone or in combination provides benefits for effectively eliminating, inhibiting, reducing, and/or preventing JCV replication and/or pathogenesis in a cell.

Further provided herein are gene editing compositions targeting a NCCR region, an early coding gene, and/or a late coding gene. In some embodiments, targeting the disclosed regions alone or in combination provides benefits for effectively eliminating, inhibiting, reducing, and/or preventing JCV replication and or pathogenesis in a cell. Provided herein, in certain embodiments, are compositions comprising (a) a Clustered Regularly Interspaced Short Palindromic Repeat (CRISPR)-associated endonuclease or a nucleic acid sequence encoding the CRISPR-associated endonuclease; (b) a first guide RNA (gRNA) or a nucleic acid sequence encoding the first gRNA, the first gRNA being complementary to a first target nucleic acid sequence within or near a non-coding control region (NCCR) of a John Cunningham virus (JCV) genome; and (c) a second gRNA or a nucleic acid sequence encoding the second gRNA, the second gRNA being complementary to a second target nucleic acid sequence within or near a T antigen gene of the JCV genome or a VP gene of the JCV genome.

Provided herein, in certain embodiments, are CRISPR-Cas systems comprising: (a) a Clustered Regularly Interspaced Short Palindromic Repeat (CRISPR)-associated endonuclease; and (b) a first guide RNA (gRNA), the first gRNA being complementary to a first target nucleic acid sequence within or near a non-coding control region (NCCR) of a John Cunningham virus (JCV) genome comprising SEQ ID NO: 2 or reverse complement thereof. In some embodiments, the CRISPR-Cas systems further comprise a second gRNA being complementary to a second target nucleic acid sequence within or near a T antigen gene of the JCV genome according to SEQ ID NO: 6. In some embodiments, the CRISPR-Cas systems further comprise a second gRNA being complementary to a second target nucleic acid sequence within or near a T antigen gene of the JCV genome comprising a sequence comprising at least about 70%, 75%, 80%, 85%, 90%, 95%, or more than 95% sequence identity to SEQ ID NO: 6. In some embodiments, the CRISPR-Cas systems further comprise a third gRNA being complementary to a third target nucleic acid sequence within or near a VP1 gene of the JCV genome according SEQ ID NO: 10. In some embodiments, the CRISPR-Cas systems further comprise a third gRNA being complementary to a third target nucleic acid sequence within or near a VP1 gene of the JCV genome comprising a sequence comprising at least about 70%, 75%, 80%, 85%, 90%, 95%, or more than 95% sequence identity to SEQ ID NO: 10.

In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 nuclease. In some embodiments, the Cas9 nuclease is a Staphylococcus aureus Cas9 nuclease. In some embodiments, the T antigen gene is a Large T antigen gene. In some embodiments, the VP gene is a VP1 gene, VP2 gene, or a VP3 gene. In some embodiments, the VP gene is a VP1 gene.

In some embodiments, first gRNA targets a NCCR sequence corresponding to SEQ ID NO: 2 or a sequence having at least 90%, 95%, 97%, 98%, or 99% sequence identity to SEQ ID NO: 2. In some embodiments, the first gRNA comprises a RNA sequence according to SEQ ID NO: 5 or a sequence having at least 90%, 95%, 97%, 98%, or 99% sequence identity to SEQ ID NO: 5. In some embodiments, the first gRNA targets a NCCR sequence complementary to SEQ ID NO: 2 or a sequence having at least 90%, 95%, 97%, 98%, or 99% sequence identity to the NCCR sequence complementary to SEQ ID NO: 2. In some embodiments, the a NCCR sequence of the NCCR targeted by the first gRNA comprises a PAM sequence according to SEQ ID NO: 3 or a sequence having at least 70%, 80%, or 90% sequence identity to SEQ ID NO: 3. In some embodiments, a NCCR sequence of the NCCR targeted by the first gRNA comprises SEQ ID NO: 4 or a sequence having at least 90%, 95%, 97%, 98%, or 99% sequence identity to SEQ ID NO: 4.

In some embodiments, the second gRNA targets a T antigen sequence corresponding to SEQ ID NO: 6 or a sequence having at least 90%, 95%, 97%, 98%, or 99% sequence identity to SEQ ID NO: 6. In some embodiments, the second gRNA comprises a RNA sequence of SEQ ID NO: 9 or a sequence having at least 90%, 95%, 97%, 98%, or 99% sequence identity to SEQ ID NO: 9. In some embodiments, the second gRNA targets a T antigen sequence complementary to SEQ ID NO: 6 or a sequence having at least 90%, 95%, 97%, 98%, or 99% sequence identity to the T antigen sequence complementary to SEQ ID NO: 6.

In some embodiments, a T antigen sequence of the T antigen gene targeted by the first gRNA comprises a PAM sequence comprising SEQ ID NO: 7 or a sequence having at least 70%, 80%, or 90% sequence identity to SEQ ID NO: 7. In some embodiments, the T antigen sequence of the T antigen gene targeted by the first gRNA comprises SEQ ID NO: 6 or a sequence having at least 90%, 95%, 97%, 98%, or 99% sequence identity to SEQ ID NO: 6. In some embodiments, the third gRNA targets a VP sequence corresponding to SEQ ID NO: 10 or a sequence having at least 90%, 95%, 97%, 98%, or 99% sequence identity to SEQ ID NO: 10. In some embodiments, the second gRNA comprises a RNA sequence of SEQ ID NO: 9 or a sequence having at least 90%, 95%, 97%, 98%, or 99% sequence identity to SEQ ID NO: 9. In some embodiments, the second gRNA targets a VP sequence complementary to SEQ ID NO: 10 or a sequence having at least 90%, 95%, 97%, 98%, or 99% sequence identity to the VP sequence complementary to SEQ ID NO: 10. In some embodiments, a VP sequence of the VP gene targeted by the first gRNA comprises a PAM sequence comprising SEQ ID NO: 10 or a sequence having at least 70%, 80%, or 90% sequence identity to SEQ ID NO: 10. In some embodiments, a VP sequence of the VP gene targeted by the first gRNA comprises SEQ ID NO: 10 or a sequence having at least 90%, 95%, 97%, 98%, or 99% sequence identity to SEQ ID NO: 10.

In some embodiments, the composition comprises a nucleic acid encoding the CRISPR-associated endonuclease, said first gRNA, and one or both of the second gRNA and third gRNA. In some embodiments, the first gRNA is encoded by SEQ ID NO: 2 or a sequence having at least 90%, 95%, 97%, 98%, or 99% sequence identity to SEQ ID NO: 2. In some embodiments, the second gRNA is encoded by SEQ ID NO: 6 or a sequence having at least 90%, 95%, 97%, 98%, or 99% sequence identity to SEQ ID NO: 6. In some embodiments, the third gRNA is encoded by SEQ ID NO: 10 or a sequence having at least 90%, 95%, 97%, 98%, or 99% sequence identity to SEQ ID NO: 10. In some embodiments, the nucleic acid encodes the first gRNA, the second gRNA, and the third gRNA. In some embodiments, the nucleic acid encodes the first gRNA, the second gRNA, and the third gRNA, wherein the first gRNA is encoded by SEQ ID NO: 2 or a sequence having at least 90%, 95%, 97%, 98%, or 99% sequence identity to SEQ ID NO: 2; the second gRNA is encoded by SEQ ID NO: 6 or a sequence having at least 90%, 95%, 97%, 98%, or 99% sequence identity to SEQ ID NO: 6; and the third gRNA is encoded by SEQ ID NO: 10 or a sequence having at least 90%, 95%, 97%, 98%, or 99% sequence identity to SEQ ID NO: 10.

Provided are nucleic acids encoding: (a) a Clustered Regularly Interspaced Short Palindromic Repeat (CRISPR)-associated endonuclease; (b) a first guide RNA (gRNA) targeting a NCCR of an JCV genome; and (c) at least one or both of a second gRNA targeting a large T antigen gene of the JCV genome and a third gRNA targeting a VP1 gene of the JCV genome. In some embodiments, the first gRNA is encoded by SEQ ID NO: 2 or a sequence having at least 90%, 95%, 97%, 98%, or 99% sequence identity to SEQ ID NO: 2. In some embodiments, the second gRNA is encoded by SEQ ID NO: 6 or a sequence having at least 90%, 95%, 97%, 98%, or 99% sequence identity to SEQ ID NO: 6. In some embodiments, the third gRNA is encoded by SEQ ID NO: 10 or a sequence having at least 90%, 95%, 97%, 98%, or 99% sequence identity to SEQ ID NO: 10.

In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 nuclease. In some embodiments, the Cas9 nuclease is a Staphylococcus aureus Cas9 nuclease.

In some embodiments, the nucleic acid further comprises a promoter. In some embodiments, the promoter is a ubiquitous promoter. In some embodiments, the promoter is a tissue-specific promoter. In some embodiments, the promoter is a constitutive promoter. In some embodiments, the promoter is a human cytomegalovirus promoter. In some embodiments, the nucleic acid further comprises an enhancer element. In some embodiments, the enhancer element is a human cytomegalovirus enhancer element.

In some embodiments, the nucleic acid further comprises a 5′ ITR element and 3′ ITR element. In some embodiments, the nucleic acid is configured to be packaged into an adeno-associated virus (AAV) vector. In some embodiments, the adeno-associated virus (AAV) vector is AAV2, AAVS, AAV6, AAV7, AAV8, or AAV9. In some embodiments, the adeno-associated virus (AAV) vector is AAV1, AAV2, AAV3, AAV4, AAVS, AAV6, AAV7, AAV8, AAV9, AAV10, AAV11, AAVDJ, or AAVDJ/8.

Further provided are adeno-associated virus (AAV) vectors comprising a nucleic acid encoding: (a) a Clustered Regularly Interspaced Short Palindromic Repeat (CRISPR)-associated endonuclease; (b) a first guide RNA (gRNA) targeting a NCCR of an JCV genome; and (c) at least one or both of a second gRNA targeting a large T antigen gene of the JCV genome and a third gRNA targeting a VP1 gene of the JCV genome. In some embodiments, the first gRNA is encoded by SEQ ID NO: 2 or a sequence having at least 90%, 95%, 97%, 98%, or 99% sequence identity to SEQ ID NO: 2. In some embodiments, the second gRNA is encoded by SEQ ID NO: 6 or a sequence having at least 90%, 95%, 97%, 98%, or 99% sequence identity to SEQ ID NO: 6. In some embodiments, the third gRNA is encoded by SEQ ID NO: 10 or a sequence having at least 90%, 95%, 97%, 98%, or 99% sequence identity to SEQ ID NO: 10.

In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 nuclease. In some embodiments, the Cas9 nuclease is a Staphylococcus aureus Cas9 nuclease. In some embodiments, the AAV vectors is an AAV6 vector or an AAV9 vector. In some embodiments, the adeno-associated virus (AAV) vector is AAV1, AAV2, AAV3, AAV4, AAVS, AAV6, AAV7, AAV8, AAV9, AAV10, AAV11, AAVDJ, or AAVDJ/8.

The compositions provided are suitable for use in a method of excising part or all of a John Cunningham virus (JCV) genome from a cell. Additionally, the compositions provided are suitable for use a method of excising part or all of a John Cunningham virus (JCV) genome from a cell. Furthermore, the compositions provided are useful in a method of reducing or eliminating expression of an infectious John Cunningham virus (JCV) in a cell. In some embodiments, method comprises reducing or eliminating expression of a Large T antigen gene, a VP1 gene, or a combination thereof. In some embodiments, the NCCR inhibits expression of one or more JCV genes and wherein the method comprises reducing or eliminating expression of one or more JCV genes. In some embodiments, editing the NCCR and editing an early coding gene and a late coding gene inhibits expression of one or more JCV genes and wherein the method comprises reducing or eliminating expression of early JCV genes and late JCV genes.

### Vectors

The present disclosure includes a vector comprising one or more cassettes for expression of CRISPR components such as one or more gRNAs and a Cas endonuclease. The vector can be any vector that is known in the art and is suitable for expressing the desired expression cassette. A number of vectors are known or can be designed to be capable of mediating transfer of gene products to mammalian cells, as is known in the art and described herein. In certain aspects, a vector refers to a nucleic acid polynucleotide to be delivered to a host cell, either in vitro or in vivo. In certain embodiments, the polynucleotide to be delivered comprises a coding sequence of interest in gene therapy (e.g. a Cas protein and gRNA). In some embodiments, a gene editing system are provided on a single vector. In some embodiments, a gene editing system are provided on a two or more vectors. In some embodiments, gene editing systems are provided by one or more vectors comprising an isolated nucleic acid encoding one or more elements of a gene editing system. In some embodiments, the CRISPR-Cas or SAM editing systems are provided by one or more vectors comprising an isolated nucleic acid encoding one or more elements of a CRISPR-Cas or SAM editing system. For example, In some embodiments, the composition comprises an isolated nucleic acid encoding a Cas protein and at least one guide nucleic acid (e.g., gRNA). In some embodiments, the composition comprises an isolated nucleic acid encoding a Cas9 protein, or functional fragment or derivative thereof. In some embodiments, the composition comprises at least one isolated nucleic acid encoding a Cas9 protein described elsewhere herein, or a functional fragment or derivative thereof. In some embodiments, the composition comprises at least one isolated nucleic acid encoding a Cas9 protein having at least 75%, 80%, 85%, 90%, 95%, 96%, 97%, 98%, or 99% amino acid sequence homology with a Cas9 protein described elsewhere herein. In certain embodiments, the isolated nucleic acid comprises any type of nucleic acid, including, but not limited to DNA and RNA. For example, In some embodiments, the composition comprises an isolated DNA molecule, including for example, an isolated cDNA molecule, encoding a gRNA or protein of the described CRISPR-Cas systems or compositions, or functional fragment thereof. In some embodiments, the composition comprises an isolated RNA molecule encoding a protein of the described CRISPR-Cas systems or compositions, or a functional fragment thereof. In certain embodiments, the isolated nucleic acids are synthesized using any method known in the art.

In some instances, the expression of natural or synthetic nucleic acids encoding a RNA and/or peptide is typically achieved by operably linking a nucleic acid encoding the RNA and/or peptide or portions thereof to a promoter and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

In some embodiments, the vectors of the present disclosure are also used for nucleic acid immunization and gene therapy, using standard gene delivery protocols. Methods for gene delivery are known in the art. In another embodiment, the disclosure provides a gene therapy vector.

The isolated nucleic acid of the disclosed can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Additional promoter elements, e.g., enhancers, regulate the frequency of transcriptional initiation. In some embodiments, the vector also includes conventional control elements which are operably linked to the transgene in a manner which permits its transcription, translation and/or expression in a cell transfected with the plasmid vector or infected with the virus comprising a nucleic acid comprising the described CRISPR-Cas systems or compositions. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and can be utilized.

Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. One example of a suitable promoter is the immediate early cytomegalovirus (CMV) promoter sequence. This promoter sequence is a strong constitutive promoter sequence capable of driving high levels of expression of any polynucleotide sequence operatively linked thereto. In certain embodiments, the Rous sarcoma virus (RSV) and MMT promoters are also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication.

Another example of a suitable promoter is Elongation Growth Factor-1α (EF-1α). However, in some embodiments, other constitutive promoter sequences are used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosed should not be limited to the use of constitutive promoters. Inducible promoters are also contemplated as part of the disclosed. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. In some instances, enhancers are located upstream or downstream of the gene it regulates. In some instances, enhancers are also tissue-specific to enhance transcription in a specific cell or tissue type. In some embodiments, the vector of the present disclosure comprises one or more enhancers to boost transcription of the gene present within the vector. In some instances, the expression of the nucleic acid and/or protein, the expression vector to be introduced into a cell can also contain either a selectable marker gene or a reporter gene or both to facilitate identification and selection of expressing cells from the population of cells sought to be transfected or infected through viral vectors. In other embodiments, the selectable marker is carried on a separate piece of DNA and used in a co-transfection procedure. Both selectable markers and reporter genes can be flanked with appropriate regulatory sequences to enable expression in the host cells. Useful selectable markers include, for example, antibiotic-resistance genes, such as neo and the like.

Provided herein, in certain embodiments, are nucleic acids that encode any of the CRISPR-Cas systems described herein. For example, provided are adeno-associated virus (AAV) vectors comprising a nucleic acid encoding: a Clustered Regularly Interspaced Short Palindromic Repeat (CRISPR)-associated endonuclease; a first guide RNA (gRNA), the first gRNA being complementary to a first target nucleic acid sequence within or near a non-coding control region (NCCR) of a John Cunningham virus (JCV) genome; and a second gRNA, the second gRNA being complementary to a second target nucleic acid sequence within or near a T antigen gene of the JCV genome or a VP gene of the JCV genome. In some embodiments, the second gRNA is complementary to a second target nucleic acid sequence within or near the T antigen gene of the JCV genome. In some embodiments, the vector further comprises a third gRNA or a nucleic acid sequence encoding the third gRNA, the third gRNA being complementary to a third target nucleic acid sequence within or near the VP gene of the JCV genome. In some embodiments, the second gRNA is complementary to a second target nucleic acid sequence within or near the VP gene of the JCV genome. In some embodiments, the CRISPR-associated endonuclease is Type I, Type II, or Type III Cas endonuclease. In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 nuclease. In some embodiments, the Cas9 nuclease is a Staphylococcus aureus Cas9 nuclease.

In some embodiments, the T antigen gene is a Large T antigen gene. In some embodiments, the VP gene is a VP1 gene, VP2 gene, or a VP3 gene. In some embodiments, the VP gene is a VP1 gene. In some embodiments, the first target nucleic acid sequence comprises a sequence comprising at least about 90% sequence identity to SEQ ID NO: 2. In some embodiments, the first target nucleic acid sequence comprises a sequence according to SEQ ID NO: 2. In some embodiments, the first target nucleic acid sequence comprises a PAM sequence comprising at least about 90% sequence identity to SEQ ID NO: 3. In some embodiments, the first target nucleic acid sequence comprises a PAM sequence according to SEQ ID NO: 3. In some embodiments, the first target nucleic acid sequence comprises a sequence comprising at least about 90% sequence identity to SEQ ID NO: 4. In some embodiments, the first target nucleic acid sequence comprises a sequence according to SEQ ID NO: 4. In some embodiments, the first gRNA is encoded by a sequence comprising at least about 90% sequence identity to SEQ ID NO: 2. In some embodiments, the first gRNA is encoded by a sequence according to SEQ ID NO: 2. In some embodiments, the first gRNA comprises a RNA sequence comprising at least about 90% sequence identity to SEQ ID NO: 5. In some embodiments, the first gRNA comprises a RNA sequence comprising SEQ ID NO: 5.

In some embodiments, the second target nucleic acid sequence comprises a sequence comprising at least about 90% sequence identity to SEQ ID NO: 6. In some embodiments, the second target nucleic acid sequence comprises a sequence comprising SEQ ID NO: 6. In some embodiments, the second target nucleic acid sequence comprises a PAM sequence comprising at least about 90% sequence identity to SEQ ID NO: 7. In some embodiments, the second target nucleic acid sequence comprises a PAM sequence comprising SEQ ID NO: 7. In some embodiments, the second target nucleic acid sequence comprises a sequence comprising at least about 90% sequence identity to SEQ ID NO: 6 In some embodiments, the second target nucleic acid sequence comprises a sequence comprising SEQ ID NO: 6. In some embodiments, the second gRNA is encoded by a sequence comprising at least about 90% sequence identity to SEQ ID NO: 6. In some embodiments, the second gRNA is encoded by a sequence according to SEQ ID NO: 6. In some embodiments, the second gRNA comprises a RNA sequence comprising at least about 90% sequence identity to SEQ ID NO: 9. In some embodiments, the second gRNA comprises a RNA sequence comprising SEQ ID NO: 9. In some embodiments, the third target nucleic acid sequence comprises a sequence comprising at least about 90% sequence identity to SEQ ID NO: 10. In some embodiments, the third target nucleic acid sequence comprises a sequence comprising SEQ ID NO: 10.

In some embodiments, the third target nucleic acid sequence comprises a PAM sequence comprising at least about 90% sequence identity to SEQ ID NO: 11. In some embodiments, the third target nucleic acid sequence comprises a PAM sequence comprising SEQ ID NO: 11. In some embodiments, the third target nucleic acid sequence comprises a sequence comprising at least about 90% sequence identity to SEQ ID NO: 12 In some embodiments, the third target nucleic acid sequence comprises a sequence comprising SEQ ID NO: 12. In some embodiments, the third gRNA is encoded by a sequence comprising at least about 90% sequence identity to SEQ ID NO: 10. In some embodiments, the third gRNA is encoded by a sequence according to SEQ ID NO: 10. In some embodiments, the third gRNA comprises a RNA sequence comprising at least about 90% sequence identity to SEQ ID NO: 13. In some embodiments, the third gRNA comprises a RNA sequence comprising SEQ ID NO: 13.

In some embodiments, the nucleic acid further comprises a promoter. In some embodiments, the promoter is a ubiquitous promoter. In some embodiments, the promoter is a tissue-specific promoter. In some embodiments, the promoter is a constitutive promoter. In some embodiments, the promoter is a human cytomegalovirus promoter. In some embodiments, the nucleic acid further comprises an enhancer element. In some embodiments, the enhancer element is a human cytomegalovirus enhancer element. In some embodiments, the nucleic acid further comprises a 5′ ITR element and 3′ ITR element.

In some embodiments, the adeno-associated virus (AAV) vector comprises an AAV2, AAVS, AAV6, AAV7, AAV8, or AAV9 capsid protein. In some embodiments, the adeno-associated virus (AAV) vector is AAV1, AAV2, AAV3, AAV4, AAVS, AAV6, AAV7, AAV8, AAV9, AAV10, AAV11, AAVDJ, or AAVDJ/8 capsid protein. In some embodiments, the nucleic acid comprising at least about 80, 85, 90, or 95% sequence identity to SEQ ID NO: 14. In some embodiments, the AAV vectors is an AAV6 vector or an AAV9 vector.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Regardless of the method used to introduce exogenous nucleic acids into a host cell, in order to confirm the presence of the recombinant nucleic acid sequence in the host cell, a variety of assays can be performed. Such assays include, for example, “molecular biological” assays well known to those of skill in the art, such as Southern and Northern blotting, RT-PCR and PCR; “biochemical” assays, such as detecting the presence or absence of a particular protein, e.g., by immunological means (ELISAs and Western blots) or by assays described herein to identify agents falling within the scope of the disclosure.

In some embodiments, a vector is provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, include but are not limited to, retroviruses, adenoviruses, adeno-associated viruses, herpes viruses, and lentiviruses. As described herein, a suitable vector generally contains an origin of replication functional in at least one organism, a promoter sequence, convenient restriction endonuclease sites, and one or more selectable markers.

Viral methods for introducing a polynucleotide of interest into a host cell include the use of DNA and RNA vectors. Viral vectors, and especially retroviral vectors, are useful for inserting genes into mammalian, e.g., human cells. Other viral vectors can be derived from lentivirus, poxviruses, herpes simplex virus I, adenoviruses and adeno-associated viruses, and the like. A number of viral based systems have been developed for gene transfer into mammalian cells. For example, retroviruses provide a convenient platform for gene delivery systems. A selected gene can be inserted into a vector and packaged in retroviral vectors using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. In another embodiment, non-AAV vectors are used, including integrating viruses, e.g., herpesvirus or lentivirus, although other viruses are selected. Suitably, where one of these other vectors is generated, it is produced as a replication-defective viral vector. In certain instances, replication-defective virus or viral vector refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In some embodiments, the genome of the viral vector does not include genes encoding the enzymes required to replicate (the genome can be engineered to be “gutless”—containing only the transgene of interest flanked by the signals required for amplification and packaging of the artificial genome), but these genes can be supplied during production.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Further provided are nucleic acids encoding the CRISPR-Cas systems described herein. Provided herein are adeno-associated virus (AAV) vectors comprising nucleic acids encoding the CRISPR-Cas systems described herein. In certain instances, an AAV vector includes to any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. In certain instances, an AAV vector includes an AAV type viral particle (or virion) comprising a nucleic acid encoding a protein of interest (e.g. CRISPR-Cas systems described herein). In some embodiments, as further described herein, the AAVs disclosed herein are be derived from various serotypes, including combinations of serotypes (e.g.,“pseudotyped” AAV) or from various genomes (e.g., single-stranded or self-complementary). In some embodiments, the AAV vector is a human serotype AAV vector. In such embodiments, a human serotype AAV is derived from any known serotype, e.g., from AAV1, AAV2, AAV4, AAV6, or AAV9. In some embodiments, the serotype is AAV1, AAV2, AAV3, AAV4, AAV5, AAV6, AAV7, AAV8, AAV9, AAV10, AAV11, AAVDJ, or AAVDJ/8.

In some embodiments, the composition includes a vector derived from an adeno-associated virus (AAV). AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

A variety of different AAV capsids have been described and can be used, although AAV which preferentially target the liver and/or deliver genes with high efficiency are particularly desired. The sequences of the AAV8 are available from a variety of databases. While the examples utilize AAV vectors having the same capsid, the capsid of the gene editing vector and the AAV targeting vector are the same AAV capsid. Another suitable AAV is, e.g., rh10 (WO 2003/042397). Still other AAV sources include, e.g., AAV9 (see, for example, U.S. Pat. No. 7,906,111; US 2011-0236353-A1), and/or hu37 (see, e.g., U.S. Pat. No. 7,906,111; US 2011-0236353-A1), AAV1, AAV2, AAV3, AAV4, AAVS, AAV6, AAV6.2, AAV7, AAV8, (U.S. Pat. Nos. 7,790,449; 7,282,199, WO 2003/042397; WO 2005/033321, WO 2006/110689; U.S. Pat. Nos. 7,790,449; 7,282,199; 7,588,772). Still other AAV can be selected, optionally taking into consideration tissue preferences of the selected AAV capsid.

In some embodiments, AAV vectors disclosed herein include a nucleic acid encoding a CRISPR-Cas systems described herein. In some embodiments, the nucleic acid also includes one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid comprises a promoter region operably linked to the coding sequence to cause or improve expression of the protein of interest in infected cells. Such a promoter can be ubiquitous, cell- or tissue-specific, strong, weak, regulated, chimeric, etc., for example, to allow efficient and stable production of the protein in the infected tissue. In certain embodiments, the promoter is homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. In certain embodiments. other promoters used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the phosphoglycerate kinase (PGK) promoter and the b-actin promoter.

In some embodiments, the recombinant AAV vector comprises packaged within an AAV capsid, a nucleic acid, generally containing a 5′ AAV ITR, the expression cassettes described herein and a 3′ AAV ITR. As described herein, in some embodiments, an expression cassette contains regulatory elements for an open reading frame(s) within each expression cassette and the nucleic acid optionally contains additional regulatory elements. The AAV vector, in some embodiments, comprises a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (see, for example, D M McCarty et al, “Self-complementary recombinant adeno-associated virus (scAAV) vectors promote efficient transduction independently of DNA synthesis”, Gene Therapy, (August 2001); see also, for example, U.S. Pat. Nos. 6,596,535; 7,125,717; and 7,456,683). Where a pseudotyped AAV is to be produced, the ITRs are selected from a source which differs from the AAV source of the capsid. For example, in some embodiments, AAV2 ITRs are selected for use with an AAV capsid having a particular efficiency for a selected cellular receptor, target tissue or viral target. In some embodiments, the ITR sequences from AAV2, or the deleted version thereof (ΔITR), are used for convenience and to accelerate regulatory approval (i.e. pseudotyped). In some embodiments, a single-stranded AAV viral vector is used.

Methods for generating and isolating AAV viral vectors suitable for delivery to a subject are known in the art (see, for example, U.S. Pat. Nos. 7,790,449; 7,282,199; WO 2003/042397; WO 2005/033321, WO 2006/110689; and U.S. Pat. Nos. 7,588,772 B2, 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors.

The CRISPR-Cas systems, for instance a Cas9, and/or any of the present RNAs, for instance a guide RNA, can be delivered using adeno associated virus (AAV), lentivirus, adenovirus or other viral vector types, or combinations thereof. Cas9 and one or more guide RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery can be either via a single dose, or multiple doses. One skilled in the art understands that the actual dosage to be delivered herein can vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

In some embodiments, dosage further contains, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, an adjuvant to enhance antigenicity, an immunostimulatory compound or molecule, and/or other compounds known in the art. The adjuvant herein can contain a suspension of minerals (alum, aluminum hydroxide, aluminum phosphate) on which antigen is adsorbed; or water-in-oil emulsion in which antigen solution is emulsified in oil (MF-59, Freund's incomplete adjuvant), sometimes with the inclusion of killed mycobacteria (Freund's complete adjuvant) to further enhance antigenicity (inhibits degradation of antigen and/or causes influx of macrophages). Adjuvants also include immunostimulatory molecules, such as cytokines, costimulatory molecules, and for example, immunostimulatory DNA or RNA molecules, such as CpG oligonucleotides. Such a dosage formulation is readily ascertainable by one skilled in the art. In some embodiments, the dosage further comprises one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. can also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. can also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in Remington's Pharmaceutical Sciences (Mack Pub. Co., N.J. 1991).

The present disclosure also provides pharmaceutical compositions comprising one or more of the compositions described herein. Formulations can be employed in admixtures with conventional excipients, i.e., pharmaceutically acceptable organic or inorganic carrier substances suitable for administration to the wound or treatment site. The pharmaceutical compositions can be sterilized and if desired mixed with auxiliary agents, e.g., lubricants, preservatives, stabilizers, wetting agents, emulsifiers, salts for influencing osmotic pressure buffers, coloring, and/or aromatic substances and the like. They can also be combined where desired with other active agents, e.g., other analgesic agents.

In some embodiments, administration of the described CRISPR-Cas systems or compositions is be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. In some embodiments, the formulations are conveniently be presented in unit dosage form, e.g. tablets and sustained release capsules, and are prepared by any methods well known in the art.

Chemical means for introducing a polynucleotide into a host cell include colloidal dispersion systems, such as macromolecule complexes, nanocapsules, nanoparticles, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid can be associated with a lipid. The nucleic acid associated with a lipid is can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The compositions described herein are suitable for use in a variety of vector systems described above. Additionally, in order to enhance the in vivo serum half-life of the administered compound, the compositions can be encapsulated, introduced into the lumen of liposomes, prepared as a colloid, or other conventional techniques can be employed which provide an extended serum half-life of the compositions (see, for example, Szoka, et al., U.S. Pat. Nos. 4,235,871, 4,501,728 and 4,837,028). Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids can assume a micellar structure or merely exist as nonuniform aggregates of lipid molecules. Also contemplated are lipofectamine-nucleic acid complexes. Methods of Treatment

The gene editing systems provided herein are useful for methods of modifying and/or editing a JCV DNA molecule (e.g. the JCV genome) in the genome of a cell (e.g. host cell). In some embodiments, targeting of the NCCR, an early coding gene (e.g. T antigen gene), and/or a late coding gene (e.g. a VP gene). In some embodiments, a gene editing system targets the NCCR. In some embodiments, a gene editing system targets a VP gene. In some embodiments, a gene editing system targets a T antigen gene. In some embodiments, a gene editing system targets the NCCR and a T antigen gene. In some embodiments, a gene editing system targets the NCCR and a VP antigen gene. In some embodiments, a gene editing system targets the NCCR, a T antigen gene, and a VP antigen gene. In some embodiments, the T antigen gene is Large T antigen. In some embodiments, the VP gene is VP1.

Provided herein, in certain embodiments, are methods of modifying and/or editing a JCV DNA molecule (e.g. the JCV genome) in the genome of a cell (e.g. host cell) using the CRISPR-Cas systems or compositions described herein. Generally, of modifying and/or editing a JCV DNA molecule (e.g. the JCV genome) in the genome of a cell (e.g. host cell) comprises contacting a cell, or providing to the cell, a CRISPR-Cas system or composition targeting the NCCR, an early coding gene (e.g. T antigen gene), and/or a late coding gene (e.g. a VP gene). In certain instances, modulating or editing a JCV DNA molecule and/or genome comprises removing and/or excising a polynucleotide sequence and/or region of the DNA molecule and/or genome. In certain instances, modulating and editing comprises removing and/or excising a polynucleotide sequence and/or region sufficient to ablate or prevent the JCV DNA molecule and/or genome from yielding a functional JCV gene product and/or a competent JCV virus (e.g. a functional JCV virus capable of replication in a host cell).

Provided herein, in certain embodiments, are methods of modulating or editing a JCV DNA comprising contacting the JCV DNA with a CRISPR-Cas system. In some embodiments, the JCV DNA is the genome of the JCV virus. In some embodiments, the JCV DNA is in a cell, wherein the cell is contacted with the CRISPR-Cas system. Further provided herein are methods of inhibiting and/or eliminating a JCV DNA in a cell comprising contacting the cell with a CRISPR-Cas system. Further provided are methods of inhibiting or preventing JCV infection and/or JCV replication in a cell comprising contacting the cell with a CRISPR-Cas systems. In some embodiments, the CRISPR-Cas system is encoded by a nucleic acid (e.g. a vector) and the cell is provided with the nucleic acid (e.g. via infection or transfection). In some embodiments, the nucleic acid is packaged in a viral vector.

In some embodiments, the CRISPR-Cas system comprises: (a) a Clustered Regularly Interspaced Short Palindromic Repeat (CRISPR)-associated endonuclease or a nucleic acid sequence encoding the CRISPR-associated endonuclease; (b) a guide RNA (gRNA) targeting (i) a non-coding control region (NCCR) of an JCV genome and/or (ii) a gRNA targeting a VP gene of the JCV genome and/or a gRNA targeting a T antigen gene of the JCV genome. In some embodiments, the CRISPR-Cas system comprises: (a) a Clustered Regularly Interspaced Short Palindromic Repeat (CRISPR)-associated endonuclease or a nucleic acid sequence encoding the CRISPR-associated endonuclease; (b) a guide RNA (gRNA) targeting a non-coding control region (NCCR) of an JCV genome; and (c) a second gRNA targeting a VP gene of the JCV genome. In some embodiments, the CRISPR-Cas system further comprises (d) a third gRNA targeting a T antigen gene of the JCV genome.

In some embodiments, the CRISPR-Cas system comprises: (a) a Clustered Regularly Interspaced Short Palindromic Repeat (CRISPR)-associated endonuclease or a nucleic acid sequence encoding the CRISPR-associated endonuclease; (b) a first guide RNA (gRNA) targeting a non-coding control region (NCCR) of an JCV genome; and (c) a second gRNA targeting a VP gene of the JCV genome. In some embodiments, the CRISPR-Cas systems further comprise (d) a third gRNA targeting a T antigen gene of the JCV genome.

In some embodiments, the CRISPR-Cas system comprises: (a) a Clustered Regularly Interspaced Short Palindromic Repeat (CRISPR)-associated endonuclease or a nucleic acid sequence encoding the CRISPR-associated endonuclease; (b) a first guide RNA (gRNA) targeting a non-coding control region (NCCR) of an JCV genome; and (c) a second gRNA targeting a Large T antigen gene of the JCV genome.

In some embodiments, the CRISPR-Cas system comprises: (a) a Clustered Regularly Interspaced Short Palindromic Repeat (CRISPR)-associated endonuclease or a nucleic acid sequence encoding the CRISPR-associated endonuclease; (b) a first guide RNA (gRNA) targeting a non-coding control region (NCCR) of an JCV genome comprising SEQ ID NO: 2 or a reverse complement thereof; and (c) a second gRNA targeting a VP gene of the JCV genome comprising SEQ ID NO: 10 or a reverse complement thereof. In some embodiments, the CRISPR-Cas systems further comprise (c) a third gRNA targeting a T antigen gene of the JCV genome comprising SEQ ID NO: 6.

In some embodiments, the CRISPR-Cas system comprises: (a) a first guide RNA (gRNA) targeting a Large T antigen gene of an JCV genome comprising SEQ ID NO: 6 or a reverse complement thereof; and (b) a second gRNA targeting a VP gene of the JCV genome comprising SEQ ID NO: 10 or a reverse complement thereof.

In some embodiments, the CRISPR-Cas system comprises: (a) a Clustered Regularly Interspaced Short Palindromic Repeat (CRISPR)-associated; and (b) a guide RNA (gRNA) targeting a sequence of a non-coding control region (NCCR) of an JCV genome comprising SEQ ID NO: 2 or reverse complement thereof. In some embodiments, the CRISPR-Cas system comprises: (a) a Clustered Regularly Interspaced Short Palindromic Repeat (CRISPR)-associated; and (b) a guide RNA (gRNA) targeting a sequence of a VP gene of an JCV genome comprising SEQ ID NO: 10 or reverse complement thereof.

Provided herein, in certain embodiments, are methods of excising part or all of a John Cunningham virus (JCV) nucleic acid from a cell, the method comprising providing to the cell a composition comprising (a) a Clustered Regularly Interspaced Short Palindromic Repeat (CRISPR)-associated endonuclease or a nucleic acid sequence encoding the CRISPR-associated endonuclease; (b) a first guide RNA (gRNA) or a nucleic acid sequence encoding the first gRNA, the first gRNA being complementary to a first target nucleic acid sequence within or near a non-coding control region (NCCR) of a John Cunningham virus (JCV) genome; and (c) a second gRNA or a nucleic acid sequence encoding the second gRNA, the second gRNA being complementary to a second target nucleic acid sequence within or near a T antigen gene of the JCV genome or a VP gene of the JCV genome. In some embodiments, the method comprises providing to the cell a CRISPR-Cas system comprising: (a) a Clustered Regularly Interspaced Short Palindromic Repeat (CRISPR)-associated endonuclease; (b) a first guide RNA (gRNA), the first gRNA being complementary to a first target nucleic acid sequence within or near a non-coding control region (NCCR) of a John Cunningham virus (JCV) genome; and (c) a second gRNA, the second gRNA being complementary to a second target nucleic acid sequence within or near a T antigen gene of the JCV genome or a VP gene of the JCV genome.

Provided herein, in certain embodiments, are methods of modulating expression of John Cunningham virus (JCV) genes in a cell, the method comprising providing a cell a composition comprising (a) a Clustered Regularly Interspaced Short Palindromic Repeat (CRISPR)-associated endonuclease or a nucleic acid sequence encoding the CRISPR-associated endonuclease; (b) a first guide RNA (gRNA) or a nucleic acid sequence encoding the first gRNA, the first gRNA being complementary to a first target nucleic acid sequence within or near a non-coding control region (NCCR) of a John Cunningham virus (JCV) genome; and (c) a second gRNA or a nucleic acid sequence encoding the second gRNA, the second gRNA being complementary to a second target nucleic acid sequence within or near a T antigen gene of the JCV genome or a VP gene of the JCV genome. In some embodiments, the method comprises providing to the cell a CRISPR-Cas system comprising: (a) a Clustered Regularly Interspaced Short Palindromic Repeat (CRISPR)-associated endonuclease; (b) a first guide RNA (gRNA), the first gRNA being complementary to a first target nucleic acid sequence within or near a non-coding control region (NCCR) of a John Cunningham virus (JCV) genome; and (c) a second gRNA, the second gRNA being complementary to a second target nucleic acid sequence within or near a T antigen gene of the JCV genome or a VP gene of the JCV genome.

In some embodiments, the first target nucleic acid sequence comprises a sequence comprising at least about 90%, 95%, 97%, 98%, or 99% sequence identity to SEQ ID NO: 2. In some embodiments, the first target nucleic acid sequence comprises a sequence according to SEQ ID NO: 2. In some embodiments, the first gRNA targets a NCCR sequence corresponding to SEQ ID NO: 2 or a sequence having at least 90%, 95%, 97%, 98%, or 99% sequence identity to SEQ ID NO: 2. In some embodiments, the first gRNA comprises a RNA sequence according to SEQ ID NO: 5 or a sequence having at least 90%, 95%, 97%, 98%, or 99% sequence identity to SEQ ID NO: 5. In some embodiments, the first gRNA targets a NCCR sequence complementary to SEQ ID NO: 2 or a sequence having at least 90%, 95%, 97%, 98%, or 99% sequence identity to the NCCR sequence complementary to SEQ ID NO: 2. In some embodiments, the gRNA comprises PAM sequence according to SEQ ID NO: 3 or a sequence having at least 70%, 80%, or 90% sequence identity to SEQ ID NO: 3. In some embodiments, a NCCR sequence of the NCCR targeted by the first gRNA comprises SEQ ID NO: 4 or a sequence having at least 90%, 95%, 97%, 98%, or 99% sequence identity to SEQ ID NO: 4.

In some embodiments, the second target nucleic acid sequence comprises a sequence comprising at least about 90%, 95%, 97%, 98%, or 99% sequence identity to SEQ ID NO: 6. In some embodiments, the second target nucleic acid sequence comprises a sequence according to SEQ ID NO: 6. In some embodiments, the second gRNA targets a T antigen sequence corresponding to SEQ ID NO: 6 or a sequence having at least 90%, 95%, 97%, 98%, or 99% sequence identity to SEQ ID NO: 6. In some embodiments, the second gRNA comprises a RNA sequence of SEQ ID NO: 9 or a sequence having at least 90%, 95%, 97%, 98%, or 99% sequence identity to SEQ ID NO: 9. In some embodiments, the second gRNA targets a T antigen sequence complementary to SEQ ID NO: 6 or a sequence having at least 90%, 95%, 97%, 98%, or 99% sequence identity to the T antigen sequence complementary to SEQ ID NO: 6. In some embodiments, a T antigen sequence of the T antigen gene targeted by the second gRNA comprises a PAM sequence comprising SEQ ID NO: 7 or a sequence having at least 70%, 80%, or 90% sequence identity to SEQ ID NO: 7. In some embodiments, a T antigen sequence of the T antigen gene targeted by the second gRNA comprises SEQ ID NO: 6 or a sequence having at least 90%, 95%, 97%, 98%, or 99% sequence identity to SEQ ID NO: 6.

In some embodiments, the third target nucleic acid sequence comprises a sequence comprising at least about 90%, 95%, 97%, 98%, or 99% sequence identity to SEQ ID NO: 10. In some embodiments, the third target nucleic acid sequence comprises a sequence according to SEQ ID NO: 10. In some embodiments, the third gRNA targets a VP sequence corresponding to SEQ ID NO: 10 or a sequence having at least 90%, 95%, 97%, 98%, or 99% sequence identity to SEQ ID NO: 10. In some embodiments, the second gRNA comprises a RNA sequence of SEQ ID NO: 9 or a sequence having at least 90%, 95%, 97%, 98%, or 99% sequence identity to SEQ ID NO: 9. In some embodiments, the second gRNA targets a VP sequence complementary to SEQ ID NO: 10 or a sequence having at least 90%, 95%, 97%, 98%, or 99% sequence identity to the VP sequence complementary to SEQ ID NO: 10. In some embodiments, a VP sequence of the VP gene targeted by the third gRNA comprises a PAM sequence comprising SEQ ID NO: 10 or a sequence having at least 70%, 80%, or 90% sequence identity to SEQ ID NO: 10. In some embodiments, a VP sequence of the VP gene targeted by the third gRNA comprises SEQ ID NO: 10 or a sequence having at least 90%, 95%, 97%, 98%, or 99% sequence identity to SEQ ID NO: 10.

In some embodiments, the CRISPR-associated endonuclease is a Cas9 endonuclease, a Cas12 endonuclease, a CasX endonuclease, or a CasΦ endonuclease. In some embodiments, the CRISPR-associated endonuclease is a Cas9 nuclease. In some embodiments, the Cas9 nuclease is a Staphylococcus aureus Cas9 nuclease.

In some embodiments, the composition comprises a nucleic acid encoding the CRISPR-associated endonuclease, said first gRNA, the second gRNA, and third gRNA. In some embodiments, the nucleic acid comprises a sequence according to SEQ ID NO: 14. In some embodiments, the nucleic acid encoding the CRISPR-associated endonuclease, said first gRNA, the second gRNA, and third gRNA is configured to be packaged in an AAV vector, wherein the AAV vector is an AAV6 vector or an AAV9 vector.

In some embodiments, the methods disclosed herein comprise providing or contacting a cell with a vector (e.g. viral or non-viral) comprising the CRISPR-Cas system of SEQ ID NO: 1 or SEQ ID NO: 14.

In some embodiments, the cell is within a subject. In some embodiments, the subject is a human. In some embodiments, the cell includes, but is not limited to, a cell within the nervous system, such as a glial cell (e.g. oligodendrocyte, astrocyte, etc.).

The methods disclosed herein further encompass, in some embodiments, administering a CRISPR-Cas system or composition. In certain instances, the pharmaceutical compositions comprising a CRISPR-Cas system or composition, as described herein, is administered. In certain instances, data obtained from the cell culture assays and animal studies can be used in formulating a range of dosage for use in humans. The dosage of such compositions lies preferably within a range of circulating concentrations that include the ED₅₀ with little or no toxicity. In some embodiments, the dosage varies within this range depending upon the dosage form employed and the route of administration utilized. For any composition used in the method of the described CRISPR-Cas systems or compositions, therapeutically effective dose can be estimated initially from cell culture assays. In certain embodiments, a dose is formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In certain instances, therapeutically effective amount and effective amount of a compound refer to an amount sufficient to provide a therapeutic benefit in the treatment, prevention and/or management of a disease, to delay or minimize one or more symptoms associated with the disease or disorder to be treated. In certain instances, therapeutically effective amount and effective amount encompass an amount that improves overall therapy, reduces or avoids symptoms or causes of disease or disorder, or enhances therapeutic efficacy of another therapeutic agent. In some embodiments, the desired dose(s) is(are) conveniently be presented in a single dose or as divided doses administered simultaneously (or over a short period of time) or at appropriate intervals, for example as two, three, four or more sub-doses per day. 101301 In some embodiments, for viral vector-mediated delivery, a dose comprises at least 1×10⁵ particles to about 1×10¹⁵ particles. In some embodiments the delivery is via an adenovirus, such as a single dose containing at least 1×10⁵ particles (also referred to as particle units, pu) of adenoviral vector. In some embodiments, the dose is at least about 1×10⁶ particles (for example, about 1×10⁶-1×10¹² particles), at least about 1×10⁷ particles, at least about 1×10⁸ particles (e.g., about 1×10⁸-1×10¹¹ particles or about 1×10⁸-1×10¹² particles), at least about 1×10⁰ particles (e.g., about 1×10⁹-1×10¹⁰ particles or about 1×10⁹-1×10¹² particles), or at least about 1×10¹⁰ particles (e.g., about 1×10-1×10¹² particles) of the adenoviral vector. Alternatively, the dose comprises no more than about 1×10¹⁴ particles, no more than about 1×10¹³ particles, no more than about 1×10¹² particles, no more than about 1×10¹¹ particles, and no more than about 1×10¹⁰ particles (e.g., no more than about 1×10⁹ particles). Thus, in some embodiments, the dose contains a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹pu, about 4×10⁹pu, about 1×10¹⁰ pu, about 2×10¹° pu, about 4×10¹⁰ pu, about 1×10¹¹pu, about 2×10¹pu, about 4×10¹¹pu, about 1×10¹²pu, about 2×10¹²pu, or about 4×10¹²pu of adenoviral vector. In some embodiments, the adenovirus is delivered via multiple doses.

In some embodiments, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage can be adjusted to balance therapeutic benefit against any side effects. In some embodiments, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹to about 1×10¹⁶ genomes AAV. In some embodiments, a human dosage is about 1×10¹³genomes AAV. In some embodiments, such concentrations are delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves (see, for example, U.S. Pat. No. 8,404,658).

In some embodiments, the cell is genetically modified in vivo in the subject in whom therapy is intended. In certain aspects, for in vivo, delivery the nucleic acid is injected directly into the subject. For example, In some embodiments, the nucleic acid is delivered at the site where the composition is required. In vivo nucleic acid transfer techniques include, but is not limited to, transfection with viral vectors such as adenovirus, Herpes simplex I virus, adeno-associated virus), lipid-based systems (useful lipids for lipid-mediated transfer of the gene are DOTMA, DOPE and DC-Chol, for example), naked DNA, and transposon-based expression systems. Exemplary gene therapy protocols see Anderson et al., Science 256:808-813 (1992). See also WO 93/25673 and the references cited therein. In some embodiments, the method comprises administering of RNA, for example mRNA, directly into the subject (see for example, Zangi et al., 2013 Nature Biotechnology, 31: 898-907).

For ex vivo treatment, an isolated cell is modified in an ex vivo or in vitro environment. In some embodiments, the cell is autologous to a subject to whom therapy is intended. Alternatively, the cell can be allogeneic, syngeneic, or xenogeneic with respect to the subject. The modified cells can then be administered to the subject directly.

One skilled in the art recognizes that different methods of delivery can be utilized to administer an isolated nucleic acid into a cell. Examples include: (1) methods utilizing physical means, such as electroporation (electricity), a gene gun (physical force) or applying large volumes of a liquid (pressure); and (2) methods wherein the nucleic acid or vector is complexed to another entity, such as a liposome, aggregated protein or transporter molecule.

The amount of vector to be added per cell will likely vary with the length and stability of therapeutic gene inserted in the vector, as well as also the nature of the sequence, and is particularly a parameter which needs to be determined empirically, and can be altered due to factors not inherent to the methods of the present disclosure (for instance, the cost associated with synthesis). One skilled in the art can easily make any necessary adjustments in accordance with the exigencies of the particular situation.

### Embodiments

Embodiment 1 comprises a composition comprising: (a) a Clustered Regularly Interspaced Short Palindromic Repeat (CRISPR)-associated endonuclease or a nucleic acid sequence encoding the CRISPR-associated endonuclease; (b) a first guide RNA (gRNA) or a nucleic acid sequence encoding the first gRNA, the first gRNA being complementary to a first target nucleic acid sequence within or near a non-coding control region (NCCR) of a John Cunningham virus (JCV) genome; and (c) a second gRNA or a nucleic acid sequence encoding the second gRNA, the second gRNA being complementary to a second target nucleic acid sequence within or near a T antigen gene of the JCV genome or a VP gene of the JCV genome. Embodiment 2 comprises a composition of embodiment 1, wherein the second gRNA is complementary to a second target nucleic acid sequence within or near the T antigen gene of the JCV genome. Embodiment 3 comprises a composition of any one of embodiments 1-2, further comprising a third gRNA or a nucleic acid sequence encoding the third gRNA, the third gRNA being complementary to a third target nucleic acid sequence within or near the VP gene of the JCV genome. Embodiment 4 comprises a composition of any one of embodiments 1-3, wherein the second gRNA is complementary to a second target nucleic acid sequence within or near the VP gene of the JCV genome. Embodiment 5 comprises a composition of any one of embodiments 1-4, wherein the CRISPR-associated endonuclease is Type I, Type II, or Type III Cas endonuclease. Embodiment 6 comprises a composition of any one of embodiments 1-5, wherein the CRISPR-associated endonuclease is a Cas9 endonuclease, a Cas12 endonuclease, a CasX endonuclease, or a CasΦ endonuclease. Embodiment 7 comprises a composition of any one of embodiments 1-6, wherein the CRISPR-associated endonuclease is a Cas9 nuclease. Embodiment 8 comprises a composition of any one of embodiments 1-7, wherein the Cas9 nuclease is a Staphylococcus aureus Cas9 nuclease. Embodiment 9 comprises a composition of any one of embodiments 1-8, wherein the T antigen gene is a Large T antigen gene. Embodiment 10 comprises a composition of any one of embodiments 1-9, wherein the VP gene is a VP1 gene, VP2 gene, or a VP3 gene. Embodiment 11 comprises a composition of any one of embodiments 1-10, wherein the VP gene is a VP1 gene. Embodiment 12 comprises a composition of any one of embodiments 1-11, wherein the first target nucleic acid sequence comprises a sequence comprising at least about 90% sequence identity to SEQ ID NO: 2. Embodiment 13 comprises a composition of any one of embodiments 1-12, wherein the first target nucleic acid sequence comprises a sequence according to SEQ ID NO: 2. Embodiment 14 comprises a composition of any one of embodiments 1-13, wherein the first target nucleic acid sequence comprises a PAM sequence comprising at least about 90% sequence identity to SEQ ID NO: 3. Embodiment 15 comprises a composition of any one of embodiments 1-14, wherein the first target nucleic acid sequence comprises a PAM sequence according to SEQ ID NO: 3. Embodiment 16 comprises a composition of any one of embodiments 1-15, wherein the first target nucleic acid sequence comprises a sequence comprising at least about 90% sequence identity to SEQ ID NO: 4. Embodiment 17 comprises a composition of any one of embodiments 1-16, wherein the first target nucleic acid sequence comprises a sequence according to SEQ ID NO: 4. Embodiment 18 comprises a composition of any one of embodiments 1-17, wherein the first gRNA is encoded by a sequence comprising at least about 90% sequence identity to SEQ ID NO: 2. Embodiment 19 comprises a composition of any one of embodiments 1-18, wherein the first gRNA is encoded by a sequence according to SEQ ID NO: 2. Embodiment 20 comprises a composition of any one of embodiments 1-19, wherein the first gRNA comprises a RNA sequence comprising at least about 90% sequence identity to SEQ ID NO: 5. Embodiment 21 comprises a composition of any one of embodiments 1-20, wherein the first gRNA comprises a RNA sequence a sequence according to SEQ ID NO: 5. Embodiment 22 comprises a composition of any one of embodiments 1-21, wherein the second target nucleic acid sequence comprises a sequence comprising at least about 90% sequence identity to SEQ ID NO: 6. Embodiment 23 comprises a composition of any one of embodiments 1-22, wherein the second target nucleic acid sequence comprises a sequence according to SEQ ID NO: 6. Embodiment 24 comprises a composition of any one of embodiments 1-23, wherein the second target nucleic acid sequence comprises a PAM sequence comprising at least about 90% sequence identity to SEQ ID NO: 7. Embodiment 25 comprises a composition of any one of embodiments 1-24, wherein the second target nucleic acid sequence comprises a PAM sequence according to SEQ ID NO: 7. Embodiment 26 comprises a composition of any one of embodiments 1-25, wherein the second target nucleic acid sequence comprises a sequence comprising at least about 90% sequence identity to SEQ ID NO: 6. Embodiment 27 comprises a composition of any one of embodiments 1-26, wherein the second target nucleic acid sequence comprises a sequence according to SEQ ID NO: 6. Embodiment 28 comprises a composition of any one of embodiments 1-27, wherein the second gRNA is encoded by a sequence comprising at least about 90% sequence identity to SEQ ID NO: 6. Embodiment 29 comprises a composition of any one of embodiments 1-28, wherein the second gRNA is encoded by a sequence according to SEQ ID NO: 6. Embodiment 30 comprises a composition of any one of embodiments 1-29, wherein the second gRNA comprises a RNA sequence comprising at least about 90% sequence identity to SEQ ID NO: 9. Embodiment 31 comprises a composition of any one of embodiments 1-30, wherein the second gRNA comprises a RNA sequence according to SEQ ID NO: 9. Embodiment 32 comprises a composition of any one of embodiments 1-31, wherein the third target nucleic acid sequence comprises a sequence comprising at least about 90% sequence identity to SEQ ID NO: 10. Embodiment 33 comprises a composition of any one of embodiments 1-32, wherein the third target nucleic acid sequence comprises a sequence according to SEQ ID NO: 10. Embodiment 34 comprises a composition of any one of embodiments 1-33, wherein the third target nucleic acid sequence comprises a PAM sequence comprising at least about 90% sequence identity to SEQ ID NO: 11. Embodiment 35 comprises a composition of any one of embodiments 1-34, wherein the third target nucleic acid sequence comprises a PAM sequence according to SEQ ID NO: 11. Embodiment 36 comprises a composition of any one of embodiments 1-35, wherein the third target nucleic acid sequence comprises a sequence comprising at least about 90% sequence identity to SEQ ID NO: 12. Embodiment 37 comprises a composition of any one of embodiments 1-36, wherein the third target nucleic acid sequence comprises a sequence according to SEQ ID NO: 12. Embodiment 38 comprises a composition of any one of embodiments 1-37, wherein the third gRNA is encoded by a sequence comprising at least about 90% sequence identity to SEQ ID NO: 10. Embodiment 39 comprises a composition of any one of embodiments 1-38, wherein the third gRNA is encoded by a sequence according to SEQ ID NO: 10. Embodiment 40 comprises a composition of any one of embodiments 1-39, wherein the third gRNA comprises a RNA sequence comprising at least about 90% sequence identity to SEQ ID NO: 13. Embodiment 41 comprises a composition of any one of embodiments 1-40, wherein the third gRNA comprises a RNA sequence according to SEQ ID NO: 13. Embodiment 42 comprises a CRISPR-Cas system comprising: (a) a Clustered Regularly Interspaced Short Palindromic Repeat (CRISPR)-associated endonuclease; and (b) a first guide RNA (gRNA), the first gRNA being complementary to a first target nucleic acid sequence within or near a non-coding control region (NCCR) of a John Cunningham virus (JCV) genome comprising SEQ ID NO: 2 or reverse complement thereof. Embodiment 43 comprises a CRISPR-Cas system of any one of embodiments 1-42, wherein the CRISPR-Cas system further comprises a second gRNA being complementary to a second target nucleic acid sequence within or near a T antigen gene of the JCV genome comprising SEQ ID NO: 6. Embodiment 44 comprises a CRISPR-Cas system of any one of embodiments 1-43, further comprising a third gRNA being complementary to a third target nucleic acid sequence within or near a VP1 gene of the JCV genome comprising SEQ ID NO: 10. Embodiment 45 comprises a CRISPR-Cas system of any one of embodiments 1-44, further comprising a second gRNA being complementary to a second target nucleic acid sequence within or near a VP1 gene of the JCV genome comprising SEQ ID NO: 10. Embodiment 46 comprises a CRISPR-Cas system comprising: (a) a Clustered Regularly Interspaced Short Palindromic Repeat (CRISPR)-associated endonuclease; and (b) a guide RNA (gRNA), the gRNA being complementary to a target nucleic acid sequence within or near a VP gene of an JCV genome comprising SEQ ID NO: 10 or reverse complement thereof. Embodiment 47 comprises a CRISPR-Cas system comprising: (a) a first guide RNA (gRNA) targeting a Large T antigen gene of an JCV genome comprising SEQ ID NO: 6 or a reverse complement thereof; and (b) a second gRNA targeting a VP gene of the JCV genome comprising SEQ ID NO: 10 or a reverse complement thereof. Embodiment 48 comprises a nucleic acid molecule encoding the CRISPR-Cas system described herein. Embodiment 49 comprises an adeno-associated virus (AAV) vector comprising a nucleic acid molecule encoding: (a) a Clustered Regularly Interspaced Short Palindromic Repeat (CRISPR)-associated endonuclease; (b) a first guide RNA (gRNA), the first gRNA being complementary to a first target nucleic acid sequence within or near a non-coding control region (NCCR) of a John Cunningham virus (JCV) genome; and (c) a second gRNA, the second gRNA being complementary to a second target nucleic acid sequence within or near a T antigen gene of the JCV genome or a VP gene of the JCV genome. Embodiment 50 comprises the AAV vector of any one of embodiments 1-49, wherein the second gRNA is complementary to a second target nucleic acid sequence within or near the T antigen gene of the JCV genome. Embodiment 51 comprises the AAV vector of any one of embodiments 1-50, wherein the AAV vectors further comprise a third gRNA or a nucleic acid sequence encoding the third gRNA, the third gRNA being complementary to a third target nucleic acid sequence within or near the VP gene of the JCV genome. Embodiment 52 comprises the AAV vector of any one of embodiments 1-51, wherein the second gRNA is complementary to a second target nucleic acid sequence within or near the VP gene of the JCV genome. Embodiment 53 comprises the AAV vector of any one of embodiments 1-52, wherein the CRISPR-associated endonuclease is Type I, Type II, or Type III Cas endonuclease. Embodiment 54 comprises the AAV vector of any one of embodiments 1-53, wherein the CRISPR-associated endonuclease is a Cas9 endonuclease, a Cas12 endonuclease, a CasX endonuclease, or a CasΦ endonuclease. Embodiment 55 comprises the AAV vector of any one of embodiments 1-54, wherein the CRISPR-associated endonuclease is a Cas9 nuclease. Embodiment 56 comprises the AAV vector of any one of embodiments 1-55, wherein the Cas9 nuclease is a Staphylococcus aureus Cas9 nuclease. Embodiment 57 comprises the AAV vector of any one of embodiments 1-56, wherein the T antigen gene is a Large T antigen gene. Embodiment 58 comprises the AAV vector of any one of embodiments 1-57, wherein the VP gene is a VP1 gene, VP2 gene, or a VP3 gene. Embodiment 59 comprises the AAV vector of any one of embodiments 1-58, wherein the VP gene is a VP1 gene. Embodiment 60 comprises the AAV vector of any one of embodiments 1-59, wherein the first target nucleic acid sequence comprises a sequence comprising at least about 90% sequence identity to SEQ ID NO: 2. Embodiment 61 comprises the AAV vector of any one of embodiments 1-60, wherein the first target nucleic acid sequence comprises a sequence according to SEQ ID NO: 2. Embodiment 62 comprises the AAV vector of any one of embodiments 1-61, wherein the first target nucleic acid sequence comprises a PAM sequence comprising at least about 90% sequence identity to SEQ ID NO: 3. Embodiment 63 comprises the AAV vector of any one of embodiments 1-62, wherein the first target nucleic acid sequence comprises a PAM sequence comprising SEQ ID NO: 3. Embodiment 64 comprises the AAV vector of any one of embodiments 1-63, wherein the first target nucleic acid sequence comprises a sequence comprising at least about 90% sequence identity to SEQ ID NO: 4. Embodiment 65 comprises the AAV vector of any one of embodiments 1-64, wherein the first target nucleic acid sequence comprises a sequence according to SEQ ID NO: 4. Embodiment 66 comprises the AAV vector of any one of embodiments 1-65, wherein the first gRNA is encoded by a sequence comprising at least about 90% sequence identity to SEQ ID NO: 2. Embodiment 67 comprises the AAV vector of any one of embodiments 1-66, wherein the first gRNA is encoded by a sequence according to SEQ ID NO: 2. Embodiment 68 comprises the AAV vector of any one of embodiments 1-67, wherein the first gRNA comprises a RNA sequence comprising at least about 90% sequence identity to SEQ ID NO: 5. Embodiment 69 comprises the AAV vector of any one of embodiments 1-68, wherein the first gRNA comprises a RNA sequence comprising SEQ ID NO: 5. Embodiment 70 comprises the AAV vector of any one of embodiments 1-69, wherein the second target nucleic acid sequence comprises a sequence comprising at least about 90% sequence identity to SEQ ID NO: 6. Embodiment 71 comprises the AAV vector of any one of embodiments 1-70, wherein the second target nucleic acid sequence comprises a sequence comprising SEQ ID NO: 6. Embodiment 72 comprises the AAV vector of any one of embodiments 1-71, wherein the second target nucleic acid sequence comprises a PAM sequence comprising at least about 90% sequence identity to SEQ ID NO: 7. Embodiment 73 comprises the AAV vector of any one of embodiments 1-72, wherein the second target nucleic acid sequence comprises a PAM sequence comprising SEQ ID NO: 7. Embodiment 74 comprises the AAV vector of any one of embodiments 1-73, wherein the second target nucleic acid sequence comprises a sequence comprising at least about 90% sequence identity to SEQ ID NO: 6. Embodiment 75 comprises the AAV vector of any one of embodiments 1-74, wherein the second target nucleic acid sequence comprises a sequence comprising SEQ ID NO: 6. Embodiment 76 comprises the AAV vector of any one of embodiments 1-75, wherein the second gRNA is encoded by a sequence comprising at least about 90% sequence identity to SEQ ID NO: 6. Embodiment 77 comprises the AAV vector of any one of embodiments 1-76, wherein the second gRNA is encoded by a sequence according to SEQ ID NO: 6. Embodiment 78 comprises the AAV vector of any one of embodiments 1-77, wherein the second gRNA comprises a RNA sequence comprising at least about 90% sequence identity to SEQ ID NO: 9. Embodiment 79 comprises the AAV vector of any one of embodiments 1-78, wherein the second gRNA comprises a RNA sequence comprising SEQ ID NO: 9. Embodiment 80 comprises the AAV vector of any one of embodiments 1-79, wherein the third target nucleic acid sequence comprises a sequence comprising at least about 90% sequence identity to SEQ ID NO: 10. Embodiment 81 comprises the AAV vector of any one of embodiments 1-80, wherein the third target nucleic acid sequence comprises a sequence comprising SEQ ID NO: 10. Embodiment 82 comprises the AAV vector of any one of embodiments 1-81, wherein the third target nucleic acid sequence comprises a PAM sequence comprising at least about 90% sequence identity to SEQ ID NO: 11. Embodiment 83 comprises the AAV vector of any one of embodiments 1-82, wherein the third target nucleic acid sequence comprises a PAM sequence comprising SEQ ID NO: 11. Embodiment 84 comprises the AAV vector of any one of embodiments 1-83, wherein the third target nucleic acid sequence comprises a sequence comprising at least about 90% sequence identity to SEQ ID NO: 12. Embodiment 85 comprises the AAV vector of any one of embodiments 1-84, wherein the third target nucleic acid sequence comprises a sequence comprising SEQ ID NO: 12. Embodiment 86 comprises the AAV vector of any one of embodiments 1-85, wherein the third gRNA is encoded by a sequence comprising at least about 90% sequence identity to SEQ ID NO: 10. Embodiment 87 comprises the AAV vector of any one of embodiments 1-86, wherein the third gRNA is encoded by a sequence according to SEQ ID NO: 10. Embodiment 88 comprises the AAV vector of any one of embodiments 1-87, wherein the third gRNA comprises a RNA sequence comprising at least about 90% sequence identity to SEQ ID NO: 13. Embodiment 89 comprises the AAV vector of any one of embodiments 1-88, wherein the third gRNA comprises a RNA sequence comprising SEQ ID NO: 13. Embodiment 90 comprises the AAV vector of any one of embodiments 1-89, wherein the nucleic acid molecule further comprises a promoter. Embodiment 91 comprises the AAV vector of any one of embodiments 1-90, wherein the promoter is a ubiquitous promoter. Embodiment 92 comprises the AAV vector of any one of embodiments 1-91, wherein the promoter is a tissue-specific promoter. Embodiment 93 comprises the AAV vector of any one of embodiments 1-92, wherein promoter is a constitutive promoter. Embodiment 94 comprises the AAV vector of any one of embodiments 1-93, wherein the promoter is a human cytomegalovirus promoter. Embodiment 95 comprises the AAV vector of any one of embodiments 1-94, wherein the nucleic acid molecule further comprises an enhancer element. Embodiment 96 comprises the AAV vector of any one of embodiments 1-95, wherein the enhancer element is a human cytomegalovirus enhancer element.

Embodiment 97 comprises the AAV vector of any one of embodiments 1-96, wherein the nucleic acid molecule further comprises a 5′ ITR element and 3′ ITR element. Embodiment 98 comprises the AAV vector of any one of embodiments 1-97, wherein the adeno-associated virus (AAV) vector is AAV2, AAVS, AAV6, AAV7, AAV8, or AAV9. Embodiment 99 comprises the AAV vector of any one of embodiments 1-98, wherein the nucleic acid molecule comprises at least about 90% sequence identity to SEQ ID NO: 14. Embodiment 100 comprises the AAV vector of any one of embodiments 1-99, wherein the AAV vectors is an AAV6 vector or an AAV9 vector. Embodiment 101 comprises a method of excising part or all of a John Cunningham virus (JCV) sequence from a cell, the method comprising providing to the cell the composition described herein, the CRISPR-Cas system described herein, or the AAV vector described herein. Embodiment 102 comprises a method of inhibiting or reducing John Cunningham virus (JCV) replication in a cell, the method comprising providing to the cell the composition described herein, the CRISPR-Cas system described herein, or the AAV vector described herein. Embodiment 103 comprises a method of any one of claims 1-102, wherein the cell is in a subject. Embodiment 104 comprises a method of any one of claims 1-103, wherein the subject is a human. Embodiment 105 comprises a method of any one of claims 1-104, wherein the JCV sequence is integrated into the cell.

### EXAMPLES

### Example 1—Inhibition of JCV by Gene Editing

Considering the limitations of current therapy, there is a need for new therapeutic approaches that not only to effectively suppress and/or inhibit JCV replication but also to eradicate latent viral genome. Accordingly, as described and provided herein, CRISPR/Cas9 systems that specifically target the JCV genome with the purpose of modulating or editing viral DNA sequences which are important for viral replication can be utilized to inhibit JCV replication. Notably, targeting the NCCR, a Large T antigen gene, and a VP1 gene of JCV drastically decreases the intact NCCR, Large T antigen gene, and VP1 gene JCV in cells, leading to suppression of JCV infection. As provided herein, the specificity of the targeting strategy (e.g. targeting the NCCR, a Large T antigen gene, and a VP1 gene) on gene editing/ablation within the JCV genome has been verified by genetic analysis of in a cell model.

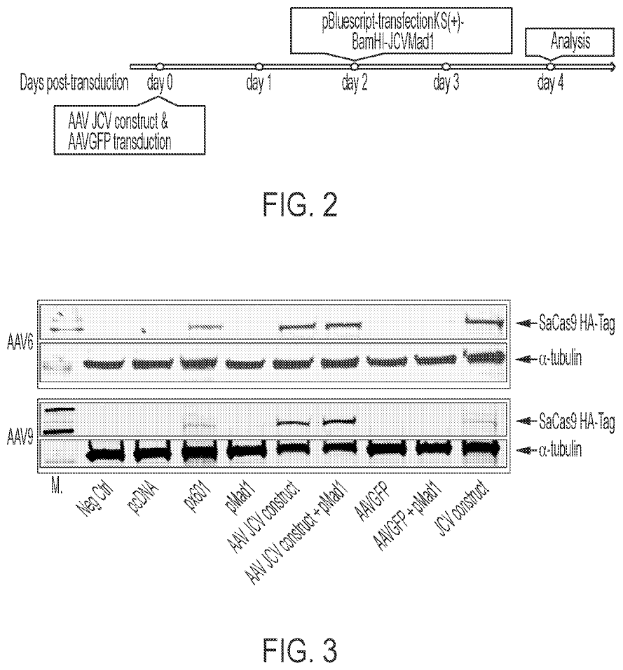

FIGS. 1A-1C show schematic representations of JCV (Mad-1) genome (FIG. 1A) and pBluescript-KS(+)-BamHI-JCVMad1 plasmid (pMad1) (FIG. 1B) with the viral genes targeted by CRISPR Cas9 gene editing system. The positions and nucleotide composition of the gRNAs including PAM (in red) are shown. The nucleotide positions are referred to the RefSeq NC_001699.1 (ncbi.nlm.nih.gov/nuccore/9628642/). FIG. 1C shows a Graphic representation of the JCV construct. The plasmid contains 3 gRNAs targeting the early gene encoding T-antigen (TAg), the late gene encoding VP1 (VP1) and the regulatory region NCCR (NCCR), respectively. Each gRNA is cloned downstream U6 promoter and 1 copy of the SaCas9 gene is also included in the plasmid. The JCV construct has been packaged in AAV6 and AAV9 vectors. FIG. 2 is a schematic representation of AAV6/AAV9 JCV construct transduction of SVGA cell line transiently transfected by the pBluescript-KS(+)-BamHI-JCVMad1 (pMad1) in order to confirm the expression of SaCas9 and the TAg-, VP1- and NCCR-related gRNAs and the excision activity on the respective gene sequence

AAV delivered Cas9 is expressed in glia cells. FIG. 3 shows a Western blot analysis confirming the expression of SaCas9 after transduction of SVGA cell line by AAV6- and AAV9-JCV construct respectively. Expression of housekeeping alpha tubulin is also shown.

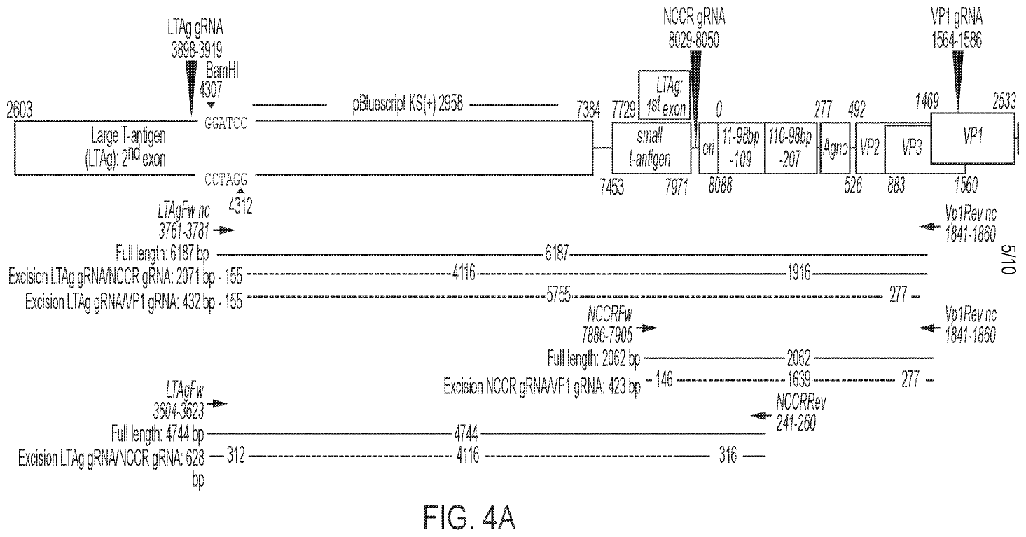

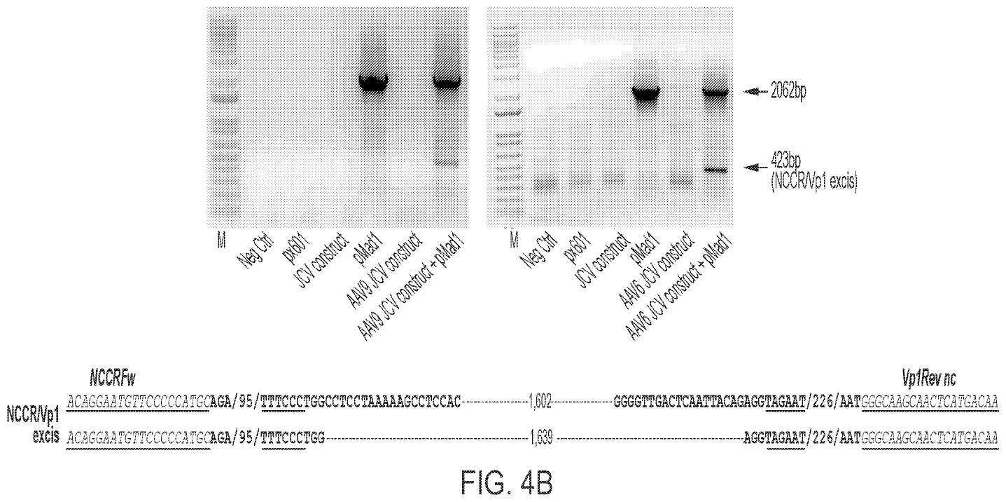

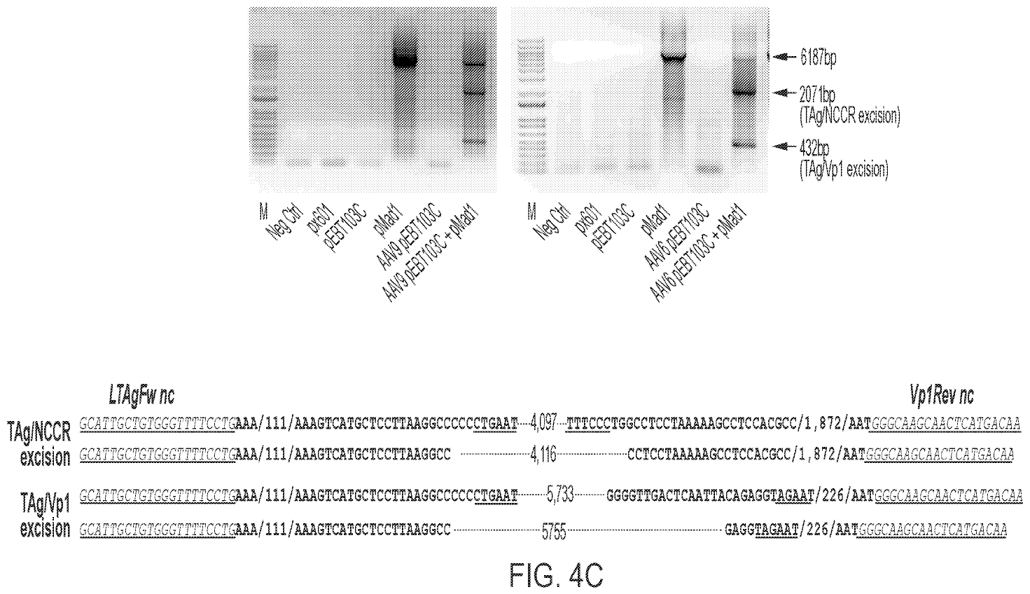

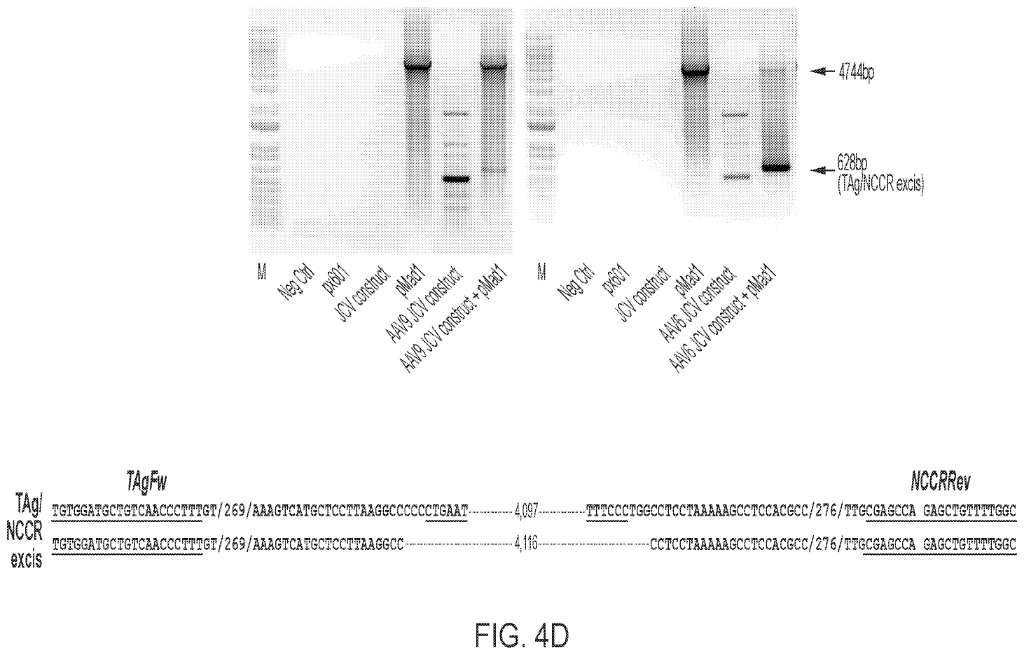

A CRISPR-Cas system having gRNAs targeting the NCCR, a Large T antigen gene, and a VP1 gene effectively excises (e.g. removes) the targeted regions for the JCV genome. \ FIGS. 4A-4D shows data from CRISPR-Cas DNA excision assays. Gel analysis illustrating amplicons obtained by JCV Mad1 specific primers in SVGA cell line transfected by pBluescript-KSH-BamHI-JCVMad1 plasmid (pMad1) after transduction with AAV6-JCV construct and AAV9-JCV construct. (FIG. 4A) Schematic representation of the position of the 3 gRNAs in the pMad1 plasmid and the specific primers used in this study in order to confirm the product of excision activity on the genome sequence of NCCR and VP1 (FIG. 4B), TAg and VP1 (FIG. 4C) and TAg and NCCR (FIGS. 4C-4D). DNA sequencing identified the specific excision induced by the specific gRNAs and SaCas9 in each target gene is also shown.

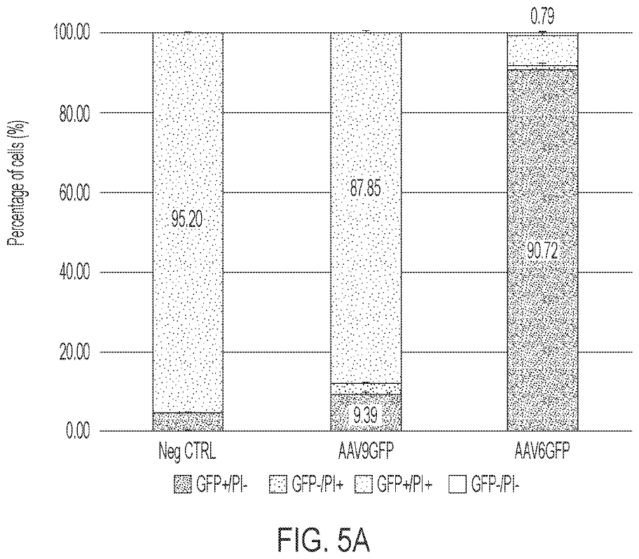

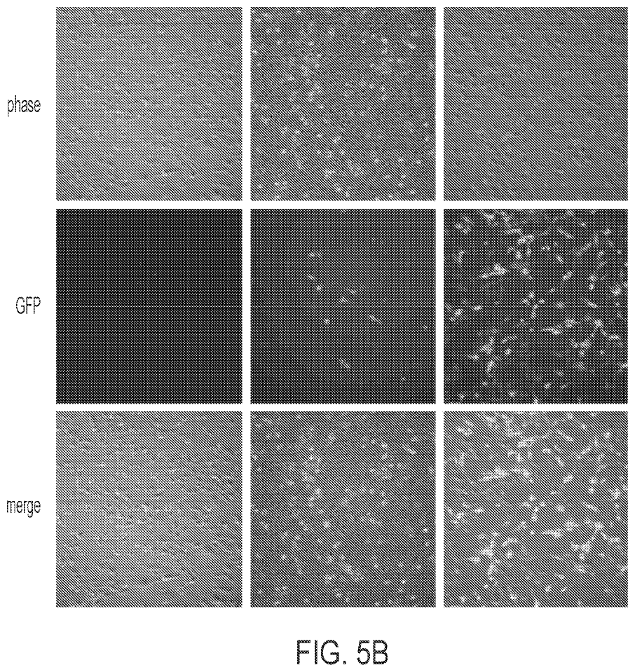

AAV6 and AAV9 effectively deliver DNA vectors to glial cells. FIGS. 5A-5B shows the efficiency of AAV6GFP and AAV9 mediated transduction into cells. in SVGA cell line. (FIG. 5A) Flow cytometry reading (after staining with propidium iodide) in order to assay the percentage of cells effectively transduced by the adeno-associated viral vectors and (FIG. 5B) Immunofluorescent evaluation of SVGA cells transduced by AAV6GFP and AAV9GFP respectively.

### Example 2. Sequences

Sequences used in the compositions and methods as described herein are provided in Table 1.

TABLE 1
Sequences

| SEQ ID NO | Sequence | Description |
|---|---|---|
| 1 | CCTGCAGGCAGCTGCGCGCTCGCTCGCTCACTGAGGCCG | JCV Construct |
| | CCCGGGCGTCGGGCGACCTTTGGTCGCCCGGCCTCAGTG | |
| | AGCGAGCGAGCGCGCAGAGAGGGAGTGGCCAACTCCATC | |
| | ACTAGGGGTTCCTGCGGCCTCTAGACTCGAGGCGTTGAC | |
| | ATTGATTATTGACTAGTTATTAATAGTAATCAATTACGGG | |
| | GTCATTAGTTCATAGCCCATATATGGAGTTCCGCGTTACA | |
| | TAACTTACGGTAAATGGCCCGCCTGGCTGACCGCCCAAC | |
| | GACCCCCGCCCATTGACGTCAATAATGACGTATGTTCCCA | |
| | TAGTAACGCCAATAGGGACTTTCCATTGACGTCAATGGGT | |
| | GGAGTATTTACGGTAAACTGCCCACTTGGCAGTACATCA | |
| | AGTGTATCATATGCCAAGTACGCCCCCTATTGACGTCAAT | |
| | GACGGTAAATGGCCCGCCTGGCATTATGCCCAGTACATG | |
| | ACCTTATGGGACTTTCCTACTTGGCAGTACATCTACGTAT | |
| | TAGTCATCGCTATTACCATGGTGATGCGGTTTTGGCAGTA | |
| | CATCAATGGGCGTGGATAGCGGTTTGACTCACGGGGATT | |
| | TCCAAGTCTCCACCCCATTGACGTCAATGGGAGTTTGTTT | |
| | TGGCACCAAAATCAACGGGACTTTCCAAAATGTCGTAAC | |
| | AACTCCGCCCCATTGACGCAAATGGGCGGTAGGCGTGTA | |
| | CGGTGGGAGGTCTATATAAGCAGAGCTCTCTGGCTAACT | |
| | ACCGGTGCCACCATGGCCCCAAAGAAGAAGCGGAAGGTC | |
| | GGTATCCACGGAGTCCCAGCAGCCAAGCGGAACTACATC | |
| | CTGGGCCTGGACATCGGCATCACCAGCGTGGGCTACGGC | |
| | ATCATCGACTACGAGACACGGGACGTGATCGATGCCGGC | |
| | GTGCGGCTGTTCAAAGAGGCCAACGTGGAAAACAACGAG | |
| | GGCAGGCGGAGCAAGAGAGGCGCCAGAAGGCTGAAGCG | |
| | GCGGAGGCGGCATAGAATCCAGAGAGTGAAGAAGCTGCT | |
| | GTTCGACTACAACCTGCTGACCGACCACAGCGAGCTGAG | |
| | CGGCATCAACCCCTACGAGGCCAGAGTGAAGGGCCTGAG | |
| | CCAGAAGCTGAGCGAGGAAGAGTTCTCTGCCGCCCTGCT | |
| | GCACCTGGCCAAGAGAAGAGGCGTGCACAACGTGAACG | |
| | AGGTGGAAGAGGACACCGGCAACGAGCTGTCCACCAAA | |
| | GAGCAGATCAGCCGGAACAGCAAGGCCCTGGAAGAGAA | |
| | ATACGTGGCCGAACTGCAGCTGGAACGGCTGAAGAAAGA | |
| | CGGCGAAGTGCGGGGCAGCATCAACAGATTCAAGACCAG | |
| | CGACTACGTGAAAGAAGCCAAACAGCTGCTGAAGGTGCA | |
| | GAAGGCCTACCACCAGCTGGACCAGAGCTTCATCGACAC | |
| | CTACATCGACCTGCTGGAAACCCGGCGGACCTACTATGA | |
| | GGGACCTGGCGAGGGCAGCCCCTTCGGCTGGAAGGACAT | |
| | CAAAGAATGGTACGAGATGCTGATGGGCCACTGCACCTA | |
| | CTTCCCCGAGGAACTGCGGAGCGTGAAGTACGCCTACAA | |
| | CGCCGACCTGTACAACGCCCTGAACGACCTGAACAATCT | |
| | CGTGATCACCAGGGACGAGAACGAGAAGCTGGAATATTA | |
| | CGAGAAGTTCCAGATCATCGAGAACGTGTTCAAGCAGAA | |
| | GAAGAAGCCCACCCTGAAGCAGATCGCCAAAGAAATCCT | |
| | CGTGAACGAAGAGGATATTAAGGGCTACAGAGTGACCAG | |
| | CACCGGCAAGCCCGAGTTCACCAACCTGAAGGTGTACCA | |
| | CGACATCAAGGACATTACCGCCCGGAAAGAGATTATTGA | |
| | GAACGCCGAGCTGCTGGATCAGATTGCCAAGATCCTGAC | |
| | CATCTACCAGAGCAGCGAGGACATCCAGGAAGAACTGAC | |
| | CAATCTGAACTCCGAGCTGACCCAGGAAGAGATCGAGCA | |
| | GATCTCTAATCTGAAGGGCTATACCGGCACCCACAACCT | |
| | GAGCCTGAAGGCCATCAACCTGATCCTGGACGAGCTGTG | |
| | GCACACCAACGACAACCAGATCGCTATCTTCAACCGGCT | |
| | GAAGCTGGTGCCCAAGAAGGTGGACCTGTCCCAGCAGAA | |
| | AGAGATCCCCACCACCCTGGTGGACGACTTCATCCTGAG | |
| | CCCCGTCGTGAAGAGAAGCTTCATCCAGAGCATCAAAGT | |
| | GATCAACGCCATCATCAAGAAGTACGGCCTGCCCAACGA | |
| | CATCATTATCGAGCTGGCCCGCGAGAAGAACTCCAAGGA | |
| | CGCCCAGAAAATGATCAACGAGATGCAGAAGCGGAACC | |
| | GGCAGACCAACGAGCGGATCGAGGAAATCATCCGGACCA | |
| | CCGGCAAAGAGAACGCCAAGTACCTGATCGAGAAGATCA | |
| | AGCTGCACGACATGCAGGAAGGCAAGTGCCTGTACAGCC | |
| | TGGAAGCCATCCCTCTGGAAGATCTGCTGAACAACCCCTT | |
| | CAACTATGAGGTGGACCACATCATCCCCAGAAGCGTGTC | |
| | CTTCGACAACAGCTTCAACAACAAGGTGCTCGTGAAGCA | |
| | GGAAGAAAACAGCAAGAAGGGCAACCGGACCCCATTCC | |
| | AGTACCTGAGCAGCAGCGACAGCAAGATCAGCTACGAAA | |
| | CCTTCAAGAAGCACATCCTGAATCTGGCCAAGGGCAAGG | |
| | GCAGAATCAGCAAGACCAAGAAAGAGTATCTGCTGGAAG | |
| | AACGGGACATCAACAGGTTCTCCGTGCAGAAAGACTTCA | |
| | TCAACCGGAACCTGGTGGATACCAGATACGCCACCAGAG | |
| | GCCTGATGAACCTGCTGCGGAGCTACTTCAGAGTGAACA | |
| | ACCTGGACGTGAAAGTGAAGTCCATCAATGGCGGCTTCA | |
| | CCAGCTTTCTGCGGCGGAAGTGGAAGTTTAAGAAAGAGC | |
| | GGAACAAGGGGTACAAGCACCACGCCGAGGACGCCCTG | |
| | ATCATTGCCAACGCCGATTTCATCTTCAAAGAGTGGAAG | |
| | AAACTGGACAAGGCCAAAAAAGTGATGGAAAACCAGAT | |
| | GTTCGAGGAAAAGCAGGCCGAGAGCATGCCCGAGATCGA | |
| | AACCGAGCAGGAGTACAAAGAGATCTTCATCACCCCCCA | |
| | CCAGATCAAGCACATTAAGGACTTCAAGGACTACAAGTA | |
| | CAGCCACCGGGTGGACAAGAAGCCTAATAGAGAGCTGAT | |
| | TAACGACACCCTGTACTCCACCCGGAAGGACGACAAGGG | |
| | CAACACCCTGATCGTGAACAATCTGAACGGCCTGTACGA | |
| | CAAGGACAATGACAAGCTGAAAAAGCTGATCAACAAGA | |
| | GCCCCGAAAAGCTGCTGATGTACCACCACGACCCCCAGA | |
| | CCTACCAGAAACTGAAGCTGATTATGGAACAGTACGGCG | |
| | ACGAGAAGAATCCCCTGTACAAGTACTACGAGGAAACCG | |
| | GGAACTACCTGACCAAGTACTCCAAAAAGGACAACGGCC | |
| | CCGTGATCAAGAAGATTAAGTATTACGGCAACAAACTGA | |
| | ACGCCCATCTGGACATCACCGACGACTACCCCAACAGCA | |
| | GAAACAAGGTCGTGAAGCTGTCCCTGAAGCCCTACAGAT | |
| | TCGACGTGTACCTGGACAATGGCGTGTACAAGTTCGTGA | |
| | CCGTGAAGAATCTGGATGTGATCAAAAAAGAAAACTACT | |
| | ACGAAGTGAATAGCAAGTGCTATGAGGAAGCTAAGAAGC | |
| | TGAAGAAGATCAGCAACCAGGCCGAGTTTATCGCCTCCT | |
| | TCTACAACAACGATCTGATCAAGATCAACGGCGAGCTGT | |
| | ATAGAGTGATCGGCGTGAACAACGACCTGCTGAACCGGA | |
| | TCGAAGTGAACATGATCGACATCACCTACCGCGAGTACC | |
| | TGGAAAACATGAACGACAAGAGGCCCCCCAGGATCATTA | |
| | AGACAATCGCCTCCAAGACCCAGAGCATTAAGAAGTACA | |
| | GCACAGACATTCTGGGCAACCTGTATGAAGTGAAATCTA | |
| | AGAAGCACCCTCAGATCATCAAAAAGGGCAAAAGGCCG | |
| | GCGGCCACGAAAAAGGCCGGCCAGGCAAAAAAGAAAAA | |
| | GGGATCCTACCCATACGATGTTCCAGATTACGCTTACCCA | |
| | TACGATGTTCCAGATTACGCTTACCCATACGATGTTCCAG | |
| | ATTACGCTTAAGAATTCCTAGAGCTCGCTGATCAGCCTCG | |
| | ACTGTGCCTTCTAGTTGCCAGCCATCTGTTGTTTGCCCCTC | |
| | CCCCGTGCCTTCCTTGACCCTGGAAGGTGCCACTCCCACT | |
| | GTCCTTTCCTAATAAAATGAGGAAATTGCATCGCATTGTC | |
| | TGAGTAGGTGTCATTCTATTCTGGGGGGTGGGGTGGGGC | |
| | AGGACAGCAAGGGGGAGGATTGGGAAGAGAATAGCAGG | |
| | CATGCTGGGGAGGTACCGAGGGCCTATTTCCCATGATTCC | |
| | TTCATATTTGCATATACGATACAAGGCTGTTAGAGAGATA | |
| | ATTGGAATTAATTTGACTGTAAACACAAAGATATTAGTAC | |
| | AAAATACGTGACGTAGAAAGTAATAATTTCTTGGGTAGT | |
| | TTGCAGTTTTAAAATTATGTTTTAAAATGGACTATCATAT | |
| | GCTTACCGTAACTTGAAAGTATTTCGATTTCTTGGCTTTAT | |
| | ATATCTTGTGGAAAGGACGAAACACCGTCATGCTCCTTA | |
| | AGGCCCCCGTTTTAGTACTCTGGAAACAGAATCTACTAAA | |
| | ACAAGGCAAAATGCCGTGTTTATCTCGTCAACTTGTTGGC | |
| | GAGATTTTTGCGGCCGCAGGAACCCCTAGTCTGAGGGCC | |
| | TATTTCCCATGATTCCTTCATATTTGCATATACGATACAA | |
| | GGCTGTTAGAGAGATAATTGGAATTAATTTGACTGTAAA | |
| | CACAAAGATATTAGTACAAAATACGTGACGTAGAAAGTA | |
| | ATAATTTCTTGGGTAGTTTGCAGTTTTAAAATTATGTTTTA | |
| | AAATGGACTATCATATGCTTACCGTAACTTGAAAGTATTT | |
| | CGATTTCTTGGCTTTATATATCTTGTGGAAAGGACGAAAC | |
| | ACCGGGGTTGACTCAATTACAGAGGGTTTTAGTACTCTGG | |
| | AAACAGAATCTACTAAAACAAGGCAAAATGCCGTGTTTA | |
| | TCTCGTCAACTTGTTGGCGAGATTTTTGCGGCCGCAGGAA | |
| | CCCCTAGTCTGAGGGCCTATTTCCCATGATTCCTTCATATT | |
| | TGCATATACGATACAAGGCTGTTAGAGAGATAATTGGAA | |
| | TTAATTTGACTGTAAACACAAAGATATTAGTACAAAATA | |
| | CGTGACGTAGAAAGTAATAATTTCTTGGGTAGTTTGCAGT | |
| | TTTAAAATTATGTTTTAAAATGGACTATCATATGCTTACC | |
| | GTAACTTGAAAGTATTTCGATTTCTTGGCTTTATATATCTT | |
| | GTGGAAAGGACGAAACACCGTGGAGGCTTTTTAGGAGGC | |
| | CAGTTTTAGTACTCTGGAAACAGAATCTACTAAAACAAG | |
| | GCAAAATGCCGTGTTTATCTCGTCAACTTGTTGGCGAGAT | |
| | TTTTGCGGCCGCAGGAACCCCTAGTGATGGAGTTGGCCA | |
| | CTCCCTCTCTGCGCGCTCGCTCGCTCACTGAGGCCGGGCG | |
| | ACCAAAGGTCGCCCGACGCCCGGGCTTTGCCCGGGCGGC | |
| | CTCAGTGAGCGAGCGAGCGCGCAGCTGCCTGCAGGGGCG | |
| | CCTGATGCGGTATTTTCTCCTTACGCATCTGTGCGGTATTT | |
| | CACACCGCATACGTCAAAGCAACCATAGTACGCGCCCTG | |
| | TAGCGGCGCATTAAGCGCGGCGGGTGTGGTGGTTACGCG | |
| | CAGCGTGACCGCTACACTTGCCAGCGCCTTAGCGCCCGCT | |
| | CCTTTCGCTTTCTTCCCTTCCTTTCTCGCCACGTTCGCCGG | |
| | CTTTCCCCGTCAAGCTCTAAATCGGGGGCTCCCTTTAGGG | |
| | TTCCGATTTAGTGCTTTACGGCACCTCGACCCCAAAAAAC | |
| | TTGATTTGGGTGATGGTTCACGTAGTGGGCCATCGCCCTG | |
| | ATAGACGGTTTTTCGCCCTTTGACGTTGGAGTCCACGTTC | |
| | TTTAATAGTGGACTCTTGTTCCAAACTGGAACAACACTCA | |
| | ACTCTATCTCGGGCTATTCTTTTGATTTATAAGGGATTTTG | |
| | CCGATTTCGGTCTATTGGTTAAAAAATGAGCTGATTTAAC | |
| | AAAAATTTAACGCGAATTTTAACAAAATATTAACGTTTAC | |
| | AATTTTATGGTGCACTCTCAGTACAATCTGCTCTGATGCC | |
| | GCATAGTTAAGCCAGCCCCGACACCCGCCAACACCCGCT | |
| | GACGCGCCCTGACGGGCTTGTCTGCTCCCGGCATCCGCTT | |
| | ACAGACAAGCTGTGACCGTCTCCGGGAGCTGCATGTGTC | |
| | AGAGGTTTTCACCGTCATCACCGAAACGCGCGAGACGAA | |
| | AGGGCCTCGTGATACGCCTATTTTTATAGGTTAATGTCAT | |
| | GATAATAATGGTTTCTTAGACGTCAGGTGGCACTTTTCGG | |
| | GGAAATGTGCGCGGAACCCCTATTTGTTTATTTTTCTAAA | |
| | TACATTCAAATATGTATCCGCTCATGAGACAATAACCCTG | |
| | ATAAATGCTTCAATAATATTGAAAAAGGAAGAGTATGAG | |
| | TATTCAACATTTCCGTGTCGCCCTTATTCCCTTTTTTGCGG | |
| | cattttgccttcctgtttttgctcacccagaaacgctggt | |
| | GAAAGTAAAAGATGCTGAAGATCAGTTGGGTGCACGAGT | |
| | GGGTTACATCGAACTGGATCTCAACAGCGGTAAGATCCT | |
| | TGAGAGTTTTCGCCCCGAAGAACGTTTTCCAATGATGAGC | |
| | acttttaaagttctgctatgtggcgcggtattatcccgta | |
| | TTGACGCCGGGCAAGAGCAACTCGGTCGCCGCATACACT | |
| | ATTCTCAGAATGACTTGGTTGAGTACTCACCAGTCACAGA | |
| | AAAGCATCTTACGGATGGCATGACAGTAAGAGAATTATG | |
| | CAGTGCTGCCATAACCATGAGTGATAACACTGCGGCCAA | |
| | CTTACTTCTGACAACGATCGGAGGACCGAAGGAGCTAAC | |
| | CGCTTTTTTGCACAACATGGGGGATCATGTAACTCGCCTT | |
| | GATCGTTGGGAACCGGAGCTGAATGAAGCCATACCAAAC | |
| | GACGAGCGTGACACCACGATGCCTGTAGCAATGGCAACA | |
| | ACGTTGCGCAAACTATTAACTGGCGAACTACTTACTCTAG | |
| | CTTCCCGGCAACAATTAATAGACTGGATGGAGGCGGATA | |
| | AAGTTGCAGGACCACTTCTGCGCTCGGCCCTTCCGGCTGG | |
| | CTGGTTTATTGCTGATAAATCTGGAGCCGGTGAGCGTGGA | |
| | AGCCGCGGTATCATTGCAGCACTGGGGCCAGATGGTAAG | |
| | CCCTCCCGTATCGTAGTTATCTACACGACGGGGAGTCAGG | |
| | CAACTATGGATGAACGAAATAGACAGATCGCTGAGATAG | |
| | GTGCCTCACTGATTAAGCATTGGTAACTGTCAGACCAAGT | |
| | TTACTCATATATACTTTAGATTGATTTAAAACTTCATTTTT | |
| | AATTTAAAAGGATCTAGGTGAAGATCCTTTTTGATAATCT | |
| | CATGACCAAAATCCCTTAACGTGAGTTTTCGTTCCACTGA | |
| | GCGTCAGACCCCGTAGAAAAGATCAAAGGATCTTCTTGA | |
| | GATCCTTTTTTTCTGCGCGTAATCTGCTGCTTGCAAACAA | |
| | AAAAACCACCGCTACCAGCGGTGGTTTGTTTGCCGGATC | |
| | AAGAGCTACCAACTCTTTTTCCGAAGGTAACTGGCTTCAG | |
| | CAGAGCGCAGATACCAAATACTGTTCTTCTAGTGTAGCCG | |
| | TAGTTAGGCCACCACTTCAAGAACTCTGTAGCACCGCCTA | |
| | CATACCTCGCTCTGCTAATCCTGTTACCAGTGGCTGCTGC | |
| | CAGTGGCGATAAGTCGTGTCTTACCGGGTTGGACTCAAG | |
| | ACGATAGTTACCGGATAAGGCGCAGCGGTCGGGCTGAAC | |
| | GGGGGGTTCGTGCACACAGCCCAGCTTGGAGCGAACGAC | |
| | CTACACCGAACTGAGATACCTACAGCGTGAGCTATGAGA | |
| | AAGCGCCACGCTTCCCGAAGGGAGAAAGGCGGACAGGT | |
| | ATCCGGTAAGCGGCAGGGTCGGAACAGGAGAGCGCACG | |
| | AGGGAGCTTCCAGGGGGAAACGCCTGGTATCTTTATAGT | |
| | CCTGTCGGGTTTCGCCACCTCTGACTTGAGCGTCGATTTT | |
| | TGTGATGCTCGTCAGGGGGGCGGAGCCTATGGAAAAACG | |
| | CCAGCAACGCGGCCTTTTTACGGTTCCTGGCCTTTTGCTG | |
| | GCCTTTTGCTCACATGT | |
| 2 | GTGGAGGCTTTTTAGGAGGCCA | NCCR Target |
| 3 | GGGAAA | NCCR PAM |
| 4 | GTGGAGGCTTTTTAGGAGGCCAGGGAAA | NCCR Target |
| | | plus PAM |
| 5 | GUGGAGGCUUUUUAGGAGGCCA | RNA |
| 6 | TCATGCTCCTTAAGGCCCCC | LTAg Target |
| 7 | CTGATT | LTAg PAM |
| 8 | TCATGCTCCTTAAGGCCCCCCTGAAT | LTAg Target |
| | | plus PAM |
| 9 | UCAUGCUCCUUAAGGCCCCC | RNA |
| 10 | GGGGTTGACTCAATTACAGAGG | VP1 Target |
| 11 | TAGAAT | VP1 PAM |
| 12 | GGGGTTGACTCAATTACAGAGGTAGAAT | VPI Target |
| | | plus PAM |
| 13 | GGGGUUGACUCAAUUACAGAGG | RNA |
| 14 | CCTGCAGGCAGCTGCGCGCTCGCTCGCTCACTGAGGCCG | AAV sequence |
| | CCCGGGCGTCGGGCGACCTTTGGTCGCCCGGCCTCAGTG | JCV construct |
| | AGCGAGCGAGCGCGCAGAGAGGGAGTGGCCAACTCCATC | |
| | ACTAGGGGTTCCTGCGGCCTCTAGACTCGAGGCGTTGAC | |
| | ATTGATTATTGACTAGTTATTAATAGTAATCAATTACGGG | |
| | GTCATTAGTTCATAGCCCATATATGGAGTTCCGCGTTACA | |
| | TAACTTACGGTAAATGGCCCGCCTGGCTGACCGCCCAAC | |
| | GACCCCCGCCCATTGACGTCAATAATGACGTATGTTCCCA | |
| | TAGTAACGCCAATAGGGACTTTCCATTGACGTCAATGGGT | |
| | GGAGTATTTACGGTAAACTGCCCACTTGGCAGTACATCA | |
| | AGTGTATCATATGCCAAGTACGCCCCCTATTGACGTCAAT | |
| | GACGGTAAATGGCCCGCCTGGCATTATGCCCAGTACATG | |
| | ACCTTATGGGACTTTCCTACTTGGCAGTACATCTACGTAT | |
| | TAGTCATCGCTATTACCATGGTGATGCGGTTTTGGCAGTA | |
| | CATCAATGGGCGTGGATAGCGGTTTGACTCACGGGGATT | |
| | TCCAAGTCTCCACCCCATTGACGTCAATGGGAGTTTGTTT | |
| | TGGCACCAAAATCAACGGGACTTTCCAAAATGTCGTAAC | |
| | AACTCCGCCCCATTGACGCAAATGGGCGGTAGGCGTGTA | |
| | CGGTGGGAGGTCTATATAAGCAGAGCTCTCTGGCTAACT | |
| | ACCGGTGCCACCATGGCCCCAAAGAAGAAGCGGAAGGTC | |
| | GGTATCCACGGAGTCCCAGCAGCCAAGCGGAACTACATC | |
| | CTGGGCCTGGACATCGGCATCACCAGCGTGGGCTACGGC | |
| | ATCATCGACTACGAGACACGGGACGTGATCGATGCCGGC | |
| | GTGCGGCTGTTCAAAGAGGCCAACGTGGAAAACAACGAG | |
| | GGCAGGCGGAGCAAGAGAGGCGCCAGAAGGCTGAAGCG | |
| | GCGGAGGCGGCATAGAATCCAGAGAGTGAAGAAGCTGCT | |
| | GTTCGACTACAACCTGCTGACCGACCACAGCGAGCTGAG | |
| | CGGCATCAACCCCTACGAGGCCAGAGTGAAGGGCCTGAG | |
| | CCAGAAGCTGAGCGAGGAAGAGTTCTCTGCCGCCCTGCT | |
| | GCACCTGGCCAAGAGAAGAGGCGTGCACAACGTGAACG | |
| | AGGTGGAAGAGGACACCGGCAACGAGCTGTCCACCAAA | |
| | GAGCAGATCAGCCGGAACAGCAAGGCCCTGGAAGAGAA | |
| | ATACGTGGCCGAACTGCAGCTGGAACGGCTGAAGAAAGA | |
| | CGGCGAAGTGCGGGGCAGCATCAACAGATTCAAGACCAG | |
| | CGACTACGTGAAAGAAGCCAAACAGCTGCTGAAGGTGCA | |
| | GAAGGCCTACCACCAGCTGGACCAGAGCTTCATCGACAC | |
| | CTACATCGACCTGCTGGAAACCCGGCGGACCTACTATGA | |
| | GGGACCTGGCGAGGGCAGCCCCTTCGGCTGGAAGGACAT | |
| | CAAAGAATGGTACGAGATGCTGATGGGCCACTGCACCTA | |
| | CTTCCCCGAGGAACTGCGGAGCGTGAAGTACGCCTACAA | |
| | CGCCGACCTGTACAACGCCCTGAACGACCTGAACAATCT | |
| | CGTGATCACCAGGGACGAGAACGAGAAGCTGGAATATTA | |
| | CGAGAAGTTCCAGATCATCGAGAACGTGTTCAAGCAGAA | |
| | GAAGAAGCCCACCCTGAAGCAGATCGCCAAAGAAATCCT | |
| | CGTGAACGAAGAGGATATTAAGGGCTACAGAGTGACCAG | |
| | CACCGGCAAGCCCGAGTTCACCAACCTGAAGGTGTACCA | |
| | CGACATCAAGGACATTACCGCCCGGAAAGAGATTATTGA | |
| | GAACGCCGAGCTGCTGGATCAGATTGCCAAGATCCTGAC | |
| | CATCTACCAGAGCAGCGAGGACATCCAGGAAGAACTGAC | |
| | CAATCTGAACTCCGAGCTGACCCAGGAAGAGATCGAGCA | |
| | GATCTCTAATCTGAAGGGCTATACCGGCACCCACAACCT | |
| | GAGCCTGAAGGCCATCAACCTGATCCTGGACGAGCTGTG | |
| | GCACACCAACGACAACCAGATCGCTATCTTCAACCGGCT | |
| | GAAGCTGGTGCCCAAGAAGGTGGACCTGTCCCAGCAGAA | |
| | AGAGATCCCCACCACCCTGGTGGACGACTTCATCCTGAG | |
| | CCCCGTCGTGAAGAGAAGCTTCATCCAGAGCATCAAAGT | |
| | GATCAACGCCATCATCAAGAAGTACGGCCTGCCCAACGA | |
| | CATCATTATCGAGCTGGCCCGCGAGAAGAACTCCAAGGA | |
| | CGCCCAGAAAATGATCAACGAGATGCAGAAGCGGAACC | |
| | GGCAGACCAACGAGCGGATCGAGGAAATCATCCGGACCA | |
| | CCGGCAAAGAGAACGCCAAGTACCTGATCGAGAAGATCA | |
| | AGCTGCACGACATGCAGGAAGGCAAGTGCCTGTACAGCC | |
| | TGGAAGCCATCCCTCTGGAAGATCTGCTGAACAACCCCTT | |
| | CAACTATGAGGTGGACCACATCATCCCCAGAAGCGTGTC | |
| | CTTCGACAACAGCTTCAACAACAAGGTGCTCGTGAAGCA | |
| | GGAAGAAAACAGCAAGAAGGGCAACCGGACCCCATTCC | |
| | AGTACCTGAGCAGCAGCGACAGCAAGATCAGCTACGAAA | |
| | CCTTCAAGAAGCACATCCTGAATCTGGCCAAGGGCAAGG | |
| | GCAGAATCAGCAAGACCAAGAAAGAGTATCTGCTGGAAG | |
| | AACGGGACATCAACAGGTTCTCCGTGCAGAAAGACTTCA | |
| | TCAACCGGAACCTGGTGGATACCAGATACGCCACCAGAG | |
| | GCCTGATGAACCTGCTGCGGAGCTACTTCAGAGTGAACA | |
| | ACCTGGACGTGAAAGTGAAGTCCATCAATGGCGGCTTCA | |
| | CCAGCTTTCTGCGGCGGAAGTGGAAGTTTAAGAAAGAGC | |
| | GGAACAAGGGGTACAAGCACCACGCCGAGGACGCCCTG | |
| | ATCATTGCCAACGCCGATTTCATCTTCAAAGAGTGGAAG | |
| | AAACTGGACAAGGCCAAAAAAGTGATGGAAAACCAGAT | |
| | GTTCGAGGAAAAGCAGGCCGAGAGCATGCCCGAGATCGA | |
| | AACCGAGCAGGAGTACAAAGAGATCTTCATCACCCCCCA | |
| | CCAGATCAAGCACATTAAGGACTTCAAGGACTACAAGTA | |
| | CAGCCACCGGGTGGACAAGAAGCCTAATAGAGAGCTGAT | |
| | TAACGACACCCTGTACTCCACCCGGAAGGACGACAAGGG | |
| | CAACACCCTGATCGTGAACAATCTGAACGGCCTGTACGA | |
| | CAAGGACAATGACAAGCTGAAAAAGCTGATCAACAAGA | |
| | GCCCCGAAAAGCTGCTGATGTACCACCACGACCCCCAGA | |
| | CCTACCAGAAACTGAAGCTGATTATGGAACAGTACGGCG | |
| | ACGAGAAGAATCCCCTGTACAAGTACTACGAGGAAACCG | |
| | GGAACTACCTGACCAAGTACTCCAAAAAGGACAACGGCC | |
| | CCGTGATCAAGAAGATTAAGTATTACGGCAACAAACTGA | |
| | ACGCCCATCTGGACATCACCGACGACTACCCCAACAGCA | |
| | GAAACAAGGTCGTGAAGCTGTCCCTGAAGCCCTACAGAT | |
| | TCGACGTGTACCTGGACAATGGCGTGTACAAGTTCGTGA | |
| | CCGTGAAGAATCTGGATGTGATCAAAAAAGAAAACTACT | |
| | ACGAAGTGAATAGCAAGTGCTATGAGGAAGCTAAGAAGC | |
| | TGAAGAAGATCAGCAACCAGGCCGAGTTTATCGCCTCCT | |
| | TCTACAACAACGATCTGATCAAGATCAACGGCGAGCTGT | |
| | ATAGAGTGATCGGCGTGAACAACGACCTGCTGAACCGGA | |
| | TCGAAGTGAACATGATCGACATCACCTACCGCGAGTACC | |
| | TGGAAAACATGAACGACAAGAGGCCCCCCAGGATCATTA | |
| | AGACAATCGCCTCCAAGACCCAGAGCATTAAGAAGTACA | |
| | GCACAGACATTCTGGGCAACCTGTATGAAGTGAAATCTA | |
| | AGAAGCACCCTCAGATCATCAAAAAGGGCAAAAGGCCG | |
| | GCGGCCACGAAAAAGGCCGGCCAGGCAAAAAAGAAAAA | |
| | GGGATCCTACCCATACGATGTTCCAGATTACGCTTACCCA | |
| | TACGATGTTCCAGATTACGCTTACCCATACGATGTTCCAG | |
| | ATTACGCTTAAGAATTCCTAGAGCTCGCTGATCAGCCTCG | |
| | ACTGTGCCTTCTAGTTGCCAGCCATCTGTTGTTTGCCCCTC | |
| | CCCCGTGCCTTCCTTGACCCTGGAAGGTGCCACTCCCACT | |
| | GTCCTTTCCTAATAAAATGAGGAAATTGCATCGCATTGTC | |
| | TGAGTAGGTGTCATTCTATTCTGGGGGGTGGGGTGGGGC | |
| | AGGACAGCAAGGGGGAGGATTGGGAAGAGAATAGCAGG | |
| | CATGCTGGGGAGGTACCGAGGGCCTATTTCCCATGATTCC | |
| | TTCATATTTGCATATACGATACAAGGCTGTTAGAGAGATA | |
| | ATTGGAATTAATTTGACTGTAAACACAAAGATATTAGTAC | |
| | AAAATACGTGACGTAGAAAGTAATAATTTCTTGGGTAGT | |
| | TTGCAGTTTTAAAATTATGTTTTAAAATGGACTATCATAT | |
| | GCTTACCGTAACTTGAAAGTATTTCGATTTCTTGGCTTTAT | |
| | ATATCTTGTGGAAAGGACGAAACACCGTCATGCTCCTTA | |
| | AGGCCCCCGTTTTAGTACTCTGGAAACAGAATCTACTAAA | |
| | ACAAGGCAAAATGCCGTGTTTATCTCGTCAACTTGTTGGC | |
| | GAGATTTTTGCGGCCGCAGGAACCCCTAGTCTGAGGGCC | |
| | TATTTCCCATGATTCCTTCATATTTGCATATACGATACAA | |
| | GGCTGTTAGAGAGATAATTGGAATTAATTTGACTGTAAA | |
| | CACAAAGATATTAGTACAAAATACGTGACGTAGAAAGTA | |
| | ATAATTTCTTGGGTAGTTTGCAGTTTTAAAATTATGTTTTA | |
| | AAATGGACTATCATATGCTTACCGTAACTTGAAAGTATTT | |
| | CGATTTCTTGGCTTTATATATCTTGTGGAAAGGACGAAAC | |
| | ACCGGGGTTGACTCAATTACAGAGGGTTTTAGTACTCTGG | |
| | AAACAGAATCTACTAAAACAAGGCAAAATGCCGTGTTTA | |
| | TCTCGTCAACTTGTTGGCGAGATTTTTGCGGCCGCAGGAA | |
| | CCCCTAGTCTGAGGGCCTATTTCCCATGATTCCTTCATATT | |
| | TGCATATACGATACAAGGCTGTTAGAGAGATAATTGGAA | |
| | TTAATTTGACTGTAAACACAAAGATATTAGTACAAAATA | |
| | CGTGACGTAGAAAGTAATAATTTCTTGGGTAGTTTGCAGT | |
| | TTTAAAATTATGTTTTAAAATGGACTATCATATGCTTACC | |
| | GTAACTTGAAAGTATTTCGATTTCTTGGCTTTATATATCTT | |
| | GTGGAAAGGACGAAACACCGTGGAGGCTTTTTAGGAGGC | |
| | CAGTTTTAGTACTCTGGAAACAGAATCTACTAAAACAAG | |
| | GCAAAATGCCGTGTTTATCTCGTCAACTTGTTGGCGAGAT | |
| | TTTTGCGGCCGCAGGAACCCCTAGTGATGGAGTTGGCCA | |
| | CTCCCTCTCTGCGCGCTCGCTCGCTCACTGAGGCCGGGCG | |
| | ACCAAAGGTCGCCCGACGCCCGGGCTTTGCCCGGGCGGC | |
| | CTCAGTGAGCGAGCGAGCGCGCAGCTGCCTGCAGGG | |

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the CRISPR-Cas systems, compositions, and methods described herein can be employed in practicing the invention. It is intended that the following claims define the scope of the disclosure and that methods and structures within the scope of these claims and their equivalents be covered thereby.

1. A composition comprising:
(a) a Clustered Regularly Interspaced Short Palindromic Repeat (CRISPR)-associated endonuclease or a nucleic acid sequence encoding the CRISPR-associated endonuclease;
(b) a first guide RNA (gRNA) or a nucleic acid sequence encoding the first gRNA, the first gRNA being complementary to a first target nucleic acid sequence within or near a non-coding control region (NCCR) of a John Cunningham virus (JCV) genome; and
(c) a second gRNA or a nucleic acid sequence encoding the second gRNA, the second gRNA being complementary to a second target nucleic acid sequence within or near a T antigen gene of the JCV genome or a VP gene of the JCV genome.

2. The composition of claim 1, wherein the second gRNA is complementary to a second target nucleic acid sequence within or near the T antigen gene of the JCV genome.

3. The composition of claim 2, further comprising a third gRNA or a nucleic acid sequence encoding the third gRNA, the third gRNA being complementary to a third target nucleic acid sequence within or near the VP gene of the JCV genome.

4. The composition of claim 1, wherein the second gRNA is complementary to a second target nucleic acid sequence within or near the VP gene of the JCV genome.

5. The composition of any one of claims 1-4, wherein the CRISPR-associated endonuclease is Type I, Type II, or Type III Cas endonuclease.

6. The composition of any one of claims 1-4, wherein the CRISPR-associated endonuclease is a Cas9 endonuclease, a Cas12 endonuclease, a CasX endonuclease, or a CasΦ endonuclease.

7. The composition of any one of claims 1-4, wherein the CRISPR-associated endonuclease is a Cas9 nuclease.

8. The composition of claim 7, wherein the Cas9 nuclease is a Staphylococcus aureus Cas9 nuclease.

9. The composition of claim 1, wherein the T antigen gene is a Large T antigen gene.

10. The composition of claim 1, wherein the VP gene is a VP1 gene, VP2 gene, or a VP3 gene.

11. The composition of claim 1, wherein the VP gene is a VP1 gene.

12. The composition of claim 1, wherein the first target nucleic acid sequence comprises a sequence comprising at least about 90% sequence identity to SEQ ID NO: 2.

13. The composition of claim 1, wherein the first target nucleic acid sequence comprises a sequence according to SEQ ID NO: 2.

14. The composition of any one of claims 1-13, wherein the first target nucleic acid sequence comprises a PAM sequence comprising at least about 90% sequence identity to SEQ ID NO: 3.

15. The composition of any one of claims 1-13, wherein the first target nucleic acid sequence comprises a PAM sequence according to SEQ ID NO: 3.

16. The composition of claim 1, wherein the first target nucleic acid sequence comprises a sequence comprising at least about 90% sequence identity to SEQ ID NO: 4.

17. The composition of claim 1, wherein the first target nucleic acid sequence comprises a sequence according to SEQ ID NO: 4.

18. The composition of claim 1, wherein the first gRNA is encoded by a sequence comprising at least about 90% sequence identity to SEQ ID NO: 2.

19. The composition of claim 1, wherein the first gRNA is encoded by a sequence according to SEQ ID NO: 2.

20. The composition of claim 1, wherein the first gRNA comprises a RNA sequence comprising at least about 90% sequence identity to SEQ ID NO: 5.

21. The composition of claim 1, wherein the first gRNA comprises a RNA sequence a sequence according to SEQ ID NO: 5.

22. The composition of claim 2, wherein the second target nucleic acid sequence comprises a sequence comprising at least about 90% sequence identity to SEQ ID NO: 6.

23. The composition of claim 2, wherein the second target nucleic acid sequence comprises a sequence according to SEQ ID NO: 6.

24. The composition of any one of claims 2-23, wherein the second target nucleic acid sequence comprises a PAM sequence comprising at least about 90% sequence identity to SEQ ID NO: 7.

25. The composition of any one of claims 2-13, wherein the second target nucleic acid sequence comprises a PAM sequence according to SEQ ID NO: 7.

26. The composition of claim 2, wherein the second target nucleic acid sequence comprises a sequence comprising at least about 90% sequence identity to SEQ ID NO: 6.

27. The composition of claim 2, wherein the second target nucleic acid sequence comprises a sequence according to SEQ ID NO: 6.

28. The composition of claim 2, wherein the second gRNA is encoded by a sequence comprising at least about 90% sequence identity to SEQ ID NO: 6.

29. The composition of claim 2, wherein the second gRNA is encoded by a sequence according to SEQ ID NO: 6.

30. The composition of claim 2, wherein the second gRNA comprises a RNA sequence comprising at least about 90% sequence identity to SEQ ID NO: 9.

31. The composition of claim 2, wherein the second gRNA comprises a RNA sequence according to SEQ ID NO: 9.

32. The composition of claim 3, wherein the third target nucleic acid sequence comprises a sequence comprising at least about 90% sequence identity to SEQ ID NO: 10.

33. The composition of claim 3, wherein the third target nucleic acid sequence comprises a sequence according to SEQ ID NO: 10.

34. The composition of any one of claims 3-33, wherein the third target nucleic acid sequence comprises a PAM sequence comprising at least about 90% sequence identity to SEQ ID NO: 11.

35. The composition of any one of claims 3-33, wherein the third target nucleic acid sequence comprises a PAM sequence according to SEQ ID NO: 11.

36. The composition of claim 3, wherein the third target nucleic acid sequence comprises a sequence comprising at least about 90% sequence identity to SEQ ID NO: 12.

37. The composition of claim 3, wherein the third target nucleic acid sequence comprises a sequence according to SEQ ID NO: 12.

38. The composition of claim 3, wherein the third gRNA is encoded by a sequence comprising at least about 90% sequence identity to SEQ ID NO: 10.

39. The composition of claim 3, wherein the third gRNA is encoded by a sequence according to SEQ ID NO: 10.

40. The composition of claim 3, wherein the third gRNA comprises a RNA sequence comprising at least about 90% sequence identity to SEQ ID NO: 13.

41. The composition of claim 3, wherein the third gRNA comprises a RNA sequence according to SEQ ID NO: 13.

42. A CRISPR-Cas system comprising:
(a) a Clustered Regularly Interspaced Short Palindromic Repeat (CRISPR)-associated endonuclease; and
(b) a first guide RNA (gRNA), the first gRNA being complementary to a first target nucleic acid sequence within or near a non-coding control region (NCCR) of a John Cunningham virus (JCV) genome comprising SEQ ID NO: 2 or reverse complement thereof.

43. The CRISPR-Cas system of claim 42, further comprising a second gRNA being complementary to a second target nucleic acid sequence within or near a T antigen gene of the JCV genome comprising SEQ ID NO: 6.

44. The CRISPR-Cas system of claim 43, further comprising a third gRNA being complementary to a third target nucleic acid sequence within or near a VP1 gene of the JCV genome comprising SEQ ID NO: 10.

45. The CRISPR-Cas system of claim 42, further comprising a second gRNA being complementary to a second target nucleic acid sequence within or near a VP1 gene of the JCV genome comprising SEQ ID NO: 10.

46. A CRISPR-Cas system comprising:
(a) a Clustered Regularly Interspaced Short Palindromic Repeat (CRISPR)-associated endonuclease; and
(b) a guide RNA (gRNA), the gRNA being complementary to a target nucleic acid sequence within or near a VP gene of an JCV genome comprising SEQ ID NO: 10 or reverse complement thereof.

47. A CRISPR-Cas system comprising:
(a) a first guide RNA (gRNA) targeting a Large T antigen gene of an JCV genome comprising SEQ ID NO: 6 or a reverse complement thereof; and
(b) a second gRNA targeting a VP gene of the JCV genome comprising SEQ ID NO: 10 or a reverse complement thereof.

48. A nucleic acid encoding the CRISPR-Cas system of any one of claims 42-47.

49. An adeno-associated virus (AAV) vector comprising a nucleic acid encoding:
(a) a Clustered Regularly Interspaced Short Palindromic Repeat (CRISPR)-associated endonuclease;
(b) a first guide RNA (gRNA), the first gRNA being complementary to a first target nucleic acid sequence within or near a non-coding control region (NCCR) of a John Cunningham virus (JCV) genome; and
(c) a second gRNA, the second gRNA being complementary to a second target nucleic acid sequence within or near a T antigen gene of the JCV genome or a VP gene of the JCV genome.

50. The AAV vector of claim 49, wherein the second gRNA is complementary to a second target nucleic acid sequence within or near the T antigen gene of the JCV genome.

51. The AAV vector of claim 50, further comprising a third gRNA or a nucleic acid sequence encoding the third gRNA, the third gRNA being complementary to a third target nucleic acid sequence within or near the VP gene of the JCV genome.

52. The AAV vector of claim 49, wherein the second gRNA is complementary to a second target nucleic acid sequence within or near the VP gene of the JCV genome.

53. The AAV vector of any one of claims 49-52, wherein the CRISPR-associated endonuclease is Type I, Type II, or Type III Cas endonuclease.

54. The AAV vector of any one of claims 49-52, wherein the CRISPR-associated endonuclease is a Cas9 endonuclease, a Cas12 endonuclease, a CasX endonuclease, or a CasΦ endonuclease.

55. The AAV vector of any one of claims 49-52, wherein the CRISPR-associated endonuclease is a Cas9 nuclease.

56. The AAV vector of claim 55, wherein the Cas9 nuclease is a Staphylococcus aureus Cas9 nuclease.

57. The AAV vector of claim 49, wherein the T antigen gene is a Large T antigen gene.

58. The AAV vector of claim 49, wherein the VP gene is a VP1 gene, VP2 gene, or a VP3 gene.

59. The AAV vector of claim 49, wherein the VP gene is a VP1 gene.

60. The AAV vector of claim 49, wherein the first target nucleic acid sequence comprises a sequence comprising at least about 90% sequence identity to SEQ ID NO: 2.

61. The AAV vector of claim 49, wherein the first target nucleic acid sequence comprises a sequence according to SEQ ID NO: 2.

62. The AAV vector of any one of claims 49-61, wherein the first target nucleic acid sequence comprises a PAM sequence comprising at least about 90% sequence identity to SEQ ID NO: 3.

63. The AAV vector of any one of claims 49-61, wherein the first target nucleic acid sequence comprises a PAM sequence according to SEQ ID NO: 3.

64. The AAV vector of claim 49, wherein the first target nucleic acid sequence comprises a sequence comprising at least about 90% sequence identity to SEQ ID NO: 4.

65. The AAV vector of claim 49, wherein the first target nucleic acid sequence comprises a sequence according to SEQ ID NO: 4.

66. The AAV vector of claim 49, wherein the first gRNA is encoded by a sequence comprising at least about 90% sequence identity to SEQ ID NO: 2.

67. The AAV vector of claim 49, wherein the first gRNA is encoded by a sequence according to SEQ ID NO: 2.

68. The AAV vector of claim 49, wherein the first gRNA comprises a RNA sequence comprising at least about 90% sequence identity to SEQ ID NO: 5.

69. The AAV vector of claim 49, wherein the first gRNA comprises a RNA sequence comprising SEQ ID NO: 5.

70. The AAV vector of claim 50, wherein the second target nucleic acid sequence comprises a sequence comprising at least about 90% sequence identity to SEQ ID NO: 6.

71. The AAV vector of claim 50, wherein the second target nucleic acid sequence comprises a sequence comprising SEQ ID NO: 6.

72. The AAV vector of any one of claims 50-71, wherein the second target nucleic acid sequence comprises a PAM sequence comprising at least about 90% sequence identity to SEQ ID NO: 7.

73. The AAV vector of any one of claims 50-71, wherein the second target nucleic acid sequence comprises a PAM sequence comprising SEQ ID NO: 7.

74. The AAV vector of claim 50, wherein the second target nucleic acid sequence comprises a sequence comprising at least about 90% sequence identity to SEQ ID NO: 6.

75. The AAV vector of claim 50, wherein the second target nucleic acid sequence comprises a sequence comprising SEQ ID NO: 6.

76. The AAV vector of claim 50, wherein the second gRNA is encoded by a sequence comprising at least about 90% sequence identity to SEQ ID NO: 6.

77. The AAV vector of claim 50, wherein the second gRNA is encoded by a sequence according to SEQ ID NO: 6.

78. The AAV vector of claim 50, wherein the second gRNA comprises a RNA sequence comprising at least about 90% sequence identity to SEQ ID NO: 9.

79. The AAV vector of claim 50, wherein the second gRNA comprises a RNA sequence comprising SEQ ID NO: 9.

80. The AAV vector of claim 51, wherein the third target nucleic acid sequence comprises a sequence comprising at least about 90% sequence identity to SEQ ID NO: 10.

81. The AAV vector of claim 51, wherein the third target nucleic acid sequence comprises a sequence comprising SEQ ID NO: 10.

82. The AAV vector of any one of claims 51-81, wherein the third target nucleic acid sequence comprises a PAM sequence comprising at least about 90% sequence identity to SEQ ID NO: 11.

83. The AAV vector of any one of claims 51-81, wherein the third target nucleic acid sequence comprises a PAM sequence comprising SEQ ID NO: 11.

84. The AAV vector of claim 51, wherein the third target nucleic acid sequence comprises a sequence comprising at least about 90% sequence identity to SEQ ID NO: 12.

85. The AAV vector of claim 51, wherein the third target nucleic acid sequence comprises a sequence comprising SEQ ID NO: 12.

86. The AAV vector of claim 51, wherein the third gRNA is encoded by a sequence comprising at least about 90% sequence identity to SEQ ID NO: 10.

87. The AAV vector of claim 51, wherein the third gRNA is encoded by a sequence according to SEQ ID NO: 10.

88. The AAV vector of claim 51, wherein the third gRNA comprises a RNA sequence comprising at least about 90% sequence identity to SEQ ID NO: 13.

89. The AAV vector of claim 51, wherein the third gRNA comprises a RNA sequence comprising SEQ ID NO: 13.

90. The AAV vector of any one of claims 49-89, wherein the nucleic acid further comprises a promoter.

91. The AAV vector of claim 90, wherein the promoter is a ubiquitous promoter.

92. The AAV vector of claim 90, wherein the promoter is a tissue-specific promoter.

93. The AAV vector of claim 90, wherein the promoter is a constitutive promoter.

94. The AAV vector of claim 90, wherein the promoter is a human cytomegalovirus promoter.

95. The AAV vector of any one of claims 49-94, wherein the nucleic acid further comprises an enhancer element.

96. The AAV vector of claim 95, wherein the enhancer element is a human cytomegalovirus enhancer element.

97. The AAV vector of any one of claims 49-96, wherein the nucleic acid further comprises a 5′ ITR element and 3′ ITR element.

98. The AAV vector of any one of claims 49-97, wherein the adeno-associated virus (AAV) vector is AAV2, AAVS, AAV6, AAV7, AAV8, or AAV9.

99. The AAV vector of any one of claims 49-98, wherein the nucleic acid comprises at least about 90% sequence identity to SEQ ID NO: 14.

100. The AAV vector of any one of claims 49-97, wherein the AAV vectors is an AAV6 vector or an AAV9 vector.

101. A method of excising part or all of a John Cunningham virus (JCV) sequence from a cell, the method comprising providing to the cell the composition of any one of claims 1-42, the CRISPR-Cas system of any one of claims 42-47, or the AAV vector of any one of claims 49-100.

102. A method of inhibiting or reducing John Cunningham virus (JCV) replication in a cell, the method comprising providing to the cell the composition of any one of claims 1-42, the CRISPR-Cas system of any one of claims 42-47, or the AAV vector of any one of claims 49-100.

103. The method of any one of claims 101-102, wherein the cell is in a subject.

104. The method of claim 103, wherein the subject is a human.

105. The method of any one of claims 101-104, wherein the JCV sequence is integrated into the cell.


SEQUENCE LISTING (25 sequences)

SEQ ID NO: 1 - DNA, 8197 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcatgctccttaaggcccccgttttagtactctggaaacagaatctactaaaacaaggcaaaatgccgtgtttatctcgtcaacttgttggcgagatttttgcggccgcaggaacccctagtctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggttgactcaattacagagggttttagtactctggaaacagaatctactaaaacaaggcaaaatgccgtgtttatctcgtcaacttgttggcgagatttttgcggccgcaggaacccctagtctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggaggctttttaggaggccagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 22 bp, Human polyomavirus 2
gtggaggctttttaggaggcca

SEQ ID NO: 3 - DNA, 6 bp, Human polyomavirus 2
gggaaa

SEQ ID NO: 4 - DNA, 28 bp, Human polyomavirus 2
gtggaggctttttaggaggccagggaaa

SEQ ID NO: 5 - RNA, 22 nt, Artificial Sequence
  Description of Artificial Sequence Synthetic oligonucleotide
guggaggcuuuuuaggaggcca

SEQ ID NO: 6 - DNA, 20 bp, Human polyomavirus 2
tcatgctccttaaggccccc

SEQ ID NO: 7 - DNA, 6 bp, Human polyomavirus 2
ctgatt

SEQ ID NO: 8 - DNA, 26 bp, Human polyomavirus 2
tcatgctccttaaggcccccctgaat

SEQ ID NO: 9 - RNA, 20 nt, Artificial Sequence
  Description of Artificial Sequence Synthetic oligonucleotide
ucaugcuccuuaaggccccc

SEQ ID NO: 10 - DNA, 22 bp, Human polyomavirus 2
ggggttgactcaattacagagg

SEQ ID NO: 11 - DNA, 6 bp, Human polyomavirus 2
tagaat

SEQ ID NO: 12 - DNA, 28 bp, Human polyomavirus 2
ggggttgactcaattacagaggtagaat

SEQ ID NO: 13 - RNA, 22 nt, Artificial Sequence
  Description of Artificial Sequence Synthetic oligonucleotide
gggguugacucaauuacagagg

SEQ ID NO: 14 - DNA, 5601 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcatgctccttaaggcccccgttttagtactctggaaacagaatctactaaaacaaggcaaaatgccgtgtttatctcgtcaacttgttggcgagatttttgcggccgcaggaacccctagtctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggttgactcaattacagagggttttagtactctggaaacagaatctactaaaacaaggcaaaatgccgtgtttatctcgtcaacttgttggcgagatttttgcggccgcaggaacccctagtctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggaggctttttaggaggccagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggg

SEQ ID NO: 15 - DNA, 23 bp, Artificial Sequence
  Description of Artificial Sequence Synthetic oligonucleotide
acaggaatgttcccccatgcaga

SEQ ID NO: 16 - DNA, 56 bp, Artificial Sequence
  Description of Artificial Sequence Synthetic oligonucleotide
tttccctggcctcctaaaaagcctccacggggttgactcaattacagaggtagaat

SEQ ID NO: 17 - DNA, 23 bp, Artificial Sequence
  Description of Artificial Sequence Synthetic oligonucleotide
aatgggcaagcaactcatgacaa

SEQ ID NO: 18 - DNA, 24 bp, Artificial Sequence
  Description of Artificial Sequence Synthetic oligonucleotide
gcattgctgtgggttttcctgaaa

SEQ ID NO: 19 - DNA, 61 bp, Artificial Sequence
  Description of Artificial Sequence Synthetic oligonucleotide
aaagtcatgctccttaaggcccccctgaattttccctggcctcctaaaaagcctccacgcc

SEQ ID NO: 20 - DNA, 43 bp, Artificial Sequence
  Description of Artificial Sequence Synthetic oligonucleotide
aaagtcatgctccttaaggcccctcctaaaaagcctccacgcc

SEQ ID NO: 21 - DNA, 58 bp, Artificial Sequence
  Description of Artificial Sequence Synthetic oligonucleotide
aaagtcatgctccttaaggcccccctgaatggggttgactcaattacagaggtagaat

SEQ ID NO: 22 - DNA, 31 bp, Artificial Sequence
  Description of Artificial Sequence Synthetic oligonucleotide
aaagtcatgctccttaaggccgaggtagaat

SEQ ID NO: 23 - DNA, 23 bp, Artificial Sequence
  Description of Artificial Sequence Synthetic oligonucleotide
tgtggatgctgtcaaccctttgt

SEQ ID NO: 24 - DNA, 23 bp, Artificial Sequence
  Description of Artificial Sequence Synthetic oligonucleotide
ttgcgagccagagctgttttggc

SEQ ID NO: 25 - DNA, 18 bp, Artificial Sequence
  Description of Artificial Sequence Synthetic oligonucleotide
tttccctggaggtagaat